(19) United States
(12) Patent Application Publication
(10) Pub. No.: US 2023/0132582 A1
(43) Pub. Date: May 4, 2023

(54) NOVEL USE OF ASPIRIN COMPOUND IN INCREASING NUCLEIC ACID EXPRESSION

(71) Applicant: SHANGHAI BELIEF-DELIVERY BIOMED CO., LTD., Shanghai (CN)

(72) Inventors: Xia WU, Palo Alto, CA (US); Xiao XIAO, Shanghai (CN); Jing ZHENG, Shanghai (CN)

(21) Appl. No.: 17/905,942

(22) PCT Filed: Mar. 10, 2021
(86) PCT No.: PCT/CN2021/079965

§ 371 Date: Sep. 9, 2022

(30) Foreign Application Priority Data
Mar. 11, 2020 (CN) PCT/CN2020/078843

(51) Int. Cl. A61K 48/00; A61K 47/12; A61K 31/713

(52) U.S. Cl. CPC A61K 48/0008; A61K 47/12; A61K 31/713

(57) ABSTRACT

The present disclosure relates to use of an aspirin compound in facilitating exogenous nucleic acid delivery and/or expression.

### TECHNICAL FIELD

The present disclosure relates to use of an aspirin compound in facilitating exogenous nucleic acid delivery and/or expression.

### BACKGROUND

Over the years genetic disorders and gene-related illness have been responsible for high mortality rates and reduced quality of life. Some of the congenital abnormalities manifest quite early, and there are minimal hopes for survival in these children, this causes much pain to their families because management option is limited and there is very little at their disposal to modify such conditions. Gene therapy is a novel form of molecular medicine, which involves transduction of full functional exogenous genes into an individual's cell or tissue to replace the defective one and modify a hereditary disease. Gene therapy has the possibility of correcting genetic disorders like hemophilia, familial hypercholesterolemia, Parkinson's disease, and Alzheimer's disease and so on.

Despite of its advantages, gene therapy also has some disadvantages such as low expression level and short-term expression. Thus, there is a need to overcome the problems in the existing exogenous gene transduction technology (especially the viral transduction technology, more preferably the AAV transduction technology) and provide a method to promote the expression of exogenous genes.

### SUMMARY OF INVENTION

In one aspect, the present disclosure provides a method of priming a cell for delivery of an exogenous nucleic acid, the method comprising: administering an aspirin compound to the cell prior to or concurrently with the delivery of the exogenous nucleic acid to the cell, wherein the exogenous nucleic acid comprises double-stranded DNA, or can be converted to double-stranded DNA in the cell after delivery.

In one aspect, the present disclosure provides a method of expressing an exogenous nucleic acid in a cell, the method comprising: delivering to the cell the exogenous nucleic acid in a condition suitable for expression, wherein the cell has been or is concurrently being administered with an aspirin compound, and wherein the exogenous nucleic acid comprises double-stranded DNA, or can be converted to double-stranded DNA in the cell after delivery.

In one aspect, the present disclosure provides a method of expressing an exogenous nucleic acid in a cell, the method comprising: a) administering an aspirin compound to the cell; and b) delivering to the cell the exogenous nucleic acid in a condition suitable for expression, wherein the step a) is prior to or concurrently with the step b), wherein the exogenous nucleic acid comprises double-stranded DNA, or can be converted to double-stranded DNA in the cell after delivery.

In one aspect, the present disclosure provides a method of increasing expression level of an exogenous nucleic acid in a cell, the method comprising: administering an aspirin compound to the cell prior to or concurrently with the delivery to the cell of the exogenous nucleic acid in a condition suitable for expression, wherein the exogenous nucleic acid comprises double-stranded DNA, or can be converted to double-stranded DNA in the cell after delivery, and whereby the expression level of the exogenous nucleic acid is increased as compared to a control expression level obtained in a control cell without the administration of the aspirin compound.

In one aspect, the present disclosure provides a method of increasing expression level of an exogenous nucleic acid in a cell, the method comprising: delivering to the cell the exogenous nucleic acid in a condition suitable for expression, wherein the cell has been or is concurrently being administered with an aspirin compound, wherein the exogenous nucleic acid comprises a double-stranded DNA, or can be converted to double-stranded DNA in the cell after delivery, and whereby the expression level of the exogenous nucleic acid is increased as compared to a control expression level obtained in a control cell without the administration of the aspirin compound.

In one aspect, the present disclosure provides a method of increasing expression level of an exogenous nucleic acid in a cell, the method comprising: a) administering an aspirin compound to the cell; and b) delivering to the cell the exogenous nucleic acid in a condition suitable for expression, wherein the step a) is prior to or concurrently with the step b), wherein the exogenous nucleic acid comprises a double-stranded DNA, or can be converted to double-stranded DNA in the cell after delivery, and wherein the expression level of the exogenous nucleic acid is increased as compared to a control expression level obtained in a control cell without the step a).

In one aspect, the present disclosure provides a method of prolonging expression duration of an exogenous nucleic acid in a cell, the method comprising: administering to the cell an aspirin compound prior to or concurrently with the delivery of the exogenous nucleic acid to the cell, wherein the exogenous nucleic acid comprises double-stranded DNA, or can be converted to double-stranded DNA in the cell after delivery, and wherein the expression duration of the exogenous nucleic acid in the cell is increased as compared to a control expression duration obtained in a control cell without the administration of the aspirin compound.

In one aspect, the present disclosure provides a method of prolonging expression duration of an exogenous nucleic acid in a cell, the method comprising: delivering to the cell the exogenous nucleic acid in a condition suitable for expression, wherein the cell has been or is concurrently being administered with an aspirin compound, wherein the exogenous nucleic acid comprises double-stranded DNA, or can be converted to double-stranded DNA in the cell after delivery, and wherein the expression duration of the exogenous nucleic acid in the cell is increased as compared to a control expression duration obtained in a control cell without the administration of the aspirin compound.

In one aspect, the present disclosure provides a method of prolonging expression duration of an exogenous nucleic acid in a cell, the method comprising: a) administering an aspirin compound to the cell; and b) delivering to the cell the exogenous nucleic acid in a condition suitable for expression, wherein the step a) is prior to or concurrently with the step b), wherein the exogenous nucleic acid comprises double-stranded DNA, or can be converted to double-stranded DNA in the cell after delivery, and whereby the expression duration of the exogenous nucleic acid is prolonged as compared to a control expression duration obtained in a control cell without being administering with aspirin compound.

In some embodiments, the cell in in vitro, ex vivo, or in vivo. In some embodiments, the aspirin compound is administered to the cell at least 1 hour, 2 hours, 3 hours, 4 hours, 5 hours, 6 hours, 7 hours, 8 hours, 9 hours, 10 hours, 12 hours, 18 hours, 24 hours, 1.5 days, 2 days, 3 days, 4 days, 5 days, 6 days or 7 days prior to the delivery of the nucleic acid. In some embodiments, the aspirin compound is administered to the cell for once or repetitively (e.g. twice, three times, four times and so on) prior to the delivery of the nucleic acid. In some embodiments, the aspirin compound is administered at an amount sufficient to provide for at least 50%, 60%, 70%, 80%, 90%, 100%, 150%, 200%, 250%, 300% or more increase in expression of the exogenous nucleic acid in the cell or in the subject. In some embodiments, the expression level is based on mRNA level or protein level. In some embodiments, the expression level is increased by at least 10%, 15%, 20%, 25%, 30%, 35%, 40%, 45%, 50%, 60%, 70%, 80%, 90%, 100%, 120%, 150%, 180%, 200%, 220%, 250%, 280%, 300%, 400%, 500%, 600%, 700%, 800%, or 900%. In some embodiments, the expression level is determined within expression duration of the exogenous nucleic acid. In some embodiments, the expression duration is the period during which the exogenous nucleic acid is expressed at a detectable level or at a physiologically effective level. In some embodiments, the expression duration is prolonged by at least 1 day, 2 days, 3 days, 4 days, 5 days, 6 days, 7 days, 8 days, 9 days or 10 days.

In some embodiments, the exogenous nucleic acid comprises a double-stranded DNA, and wherein the double-stranded DNA comprises a double-stranded DNA virus vector, a double-stranded plasmid, or a double-stranded artificial chromosome. In some embodiments, the exogenous nucleic acid can be converted to double-stranded DNA after delivery to a cell or a subject, and wherein the exogenous nucleic acid comprises a single strand DNA, a retrovirus vector, or a lentivirus vector. In some embodiments, the exogenous nucleic acid comprises or is contained within a viral vector (e.g. an adeno-associated virus (AAV) vector, lentivirus vector, retrovirus vector, or adenovirus vector), plasmid, or exosomes. In some embodiments, the viral vector comprises an adeno-associated virus (AAV) vector. In some embodiments, the AAV vector comprises an AAV virus particle. In some embodiments, the AAV vector comprises a cap gene encoding a capsid protein. In some embodiments, the AAV vector comprises an AAV virus particle comprising a native or recombinant capsid protein. In some embodiments, the capsid protein can be modified or chimeric or synthetic. In some embodiments, the cap gene or the capsid protein is derived from two or more AAV serotypes. In some embodiments, the cap gene or the capsid protein can have a specific tropism profile.

In some embodiments, the exogenous nucleic acid comprises an encoding sequence that encodes for a protein of interest, or a portion thereof, or that encodes for a functional RNA or a portion thereof. In some embodiments, the protein of interest comprises a therapeutic protein an immunogenic protein, a reporter protein, a nuclease or a therapeutic target protein, and/or the functional RNA comprises an antisense oligonucleotide, ribozyme, RNAs that effect spliceosome-mediated/raw-splicing, interfering RNAs (RNAi), or other non-translated functional RNAs, such as guide RNAs and single guide RNAs. In some embodiments, the exogenous nucleic acid is delivered to a subject or a cell in a condition suitable for expression. In some embodiments, the encoding sequence is operably linked to one or more regulatory sequences.

In another aspect, the present disclosure provides a method of priming a subject having a condition treatable by an exogenous nucleic acid or the expression product thereof, the method comprising: administering an effective amount of an aspirin compound to the subject prior to or concurrently with the delivery of the exogenous nucleic acid to the cell, wherein the exogenous nucleic acid of the present disclosure comprises double-stranded DNA, or can be converted to double-stranded DNA in the subject after delivery.

In another aspect, the present disclosure provides a method of treating or preventing a condition treatable or preventable by an exogenous nucleic acid or the expression product thereof, the method comprising: delivering to the subject a therapeutically effective amount of the exogenous nucleic acid, wherein the exogenous nucleic acid comprises double-stranded DNA, or can be converted to double-stranded DNA in the subject after delivery, and wherein the subject has been or is concurrently being administered with an aspirin compound.

In another aspect, the present disclosure provides a method of treating or preventing a condition treatable or preventable by an exogenous nucleic acid or the expression product thereof, the method comprising: a) administering an effective amount of an aspirin compound to the subject; and b) delivering to the subject a therapeutically effective amount of the exogenous nucleic acid, wherein the step a) is prior to or concurrently with the step b), wherein the exogenous nucleic acid comprises double-stranded DNA, or can be converted to double-stranded DNA in the cell after delivery.

In some embodiments, the condition is characterized by deficiency of one or more functional genes or functional protein(s). In some embodiments, the condition is a single gene disorder. In some embodiments, the single gene disorder is an autosomal dominant, autosomal recessive, X-linked, Y-linked or mitochondrial.

In certain embodiments, the condition treatable is a CNS disorder. In certain embodiments, the CNS disorder is selected from the group consisting of Parkinson's disease, Alzheimer's disease, Mucopolysaccharidosis type II, Mucopolysaccharidosis type IIIA, Mucopolysaccharidosis type IIIB, Huntington disease, amyotrophic lateral sclerosis, Epilepsy, Batten Disease, Spinocerebellar Ataxia, spinal muscular atrophy, Canavan disease, and Friedreich's ataxia.

In certain embodiments, the exogenous nucleic acid comprises sequence encoding for a protein of interest or a portion thereof, wherein the protein of interest is selected from the group consisting of Tau, MeCP2, NGF, APOE, GDNF, SUMF, SGSH, AADC, CD, p53, ARSA arylsulfatase A, ABCD1, SMN1, NAGLU, SOD1, C9ORF72, TARDBP, FUS, HTT, LRRK2, PARIS, PARKIN, GAD, and α-synuclein. In some embodiments, the exogenous nucleic acid comprises an AAV vector, optionally comprising an AAV virus particle. In certain embodiments, the exogenous nucleic acid comprises an AAV vector of AAV9 serotype (e.g. an AAV virus particle of AAV9 serotype). In some embodiments, the therapeutically effective amount ranges from 10⁶ to 10¹⁴ vg/kg (vector genomes/kg). In certain embodiments, therapeutic effective amount is no more than 10¹⁴ vg/kg (e.g. no more 10¹³ vg/kg, 10^{12.5} vg/kg, 10¹² vg/kg, 10¹¹ vg/kg or even lower.

In certain embodiments, the aspirin compound and/or the exogenous nucleic acid is administered via a systemic administration (e.g. intravenous, intramuscular, subcutaneous administration), or via intraparenchymal, intracerebroventricular, or intrathecal routes.

In some embodiments, the therapeutically effective amount is a sub-therapeutic amount.

In another aspect, the present disclosure provides a method of reducing adverse effects or improving tolerance to an exogenous nucleic acid in a subject, the method comprising: delivering to the subject a sub-therapeutic amount of the exogenous nucleic acid for treating or preventing a condition, wherein the exogenous nucleic acid comprises double-stranded DNA, or can be converted to double-stranded DNA in the subject after delivery, and wherein the subject has been or is concurrently being administered with an effective amount of an aspirin compound.

In another aspect, the present disclosure provides a method of reducing adverse effects or improving tolerance to an exogenous nucleic acid in a subject, the method comprising: a) administering an effective amount of an aspirin compound to the subject; and b) delivering to the subject a sub-therapeutic amount of the exogenous nucleic acid for treating or preventing a condition, wherein the step a) is prior to or concurrently with the step b), wherein the exogenous nucleic acid comprises double-stranded DNA, or can be converted to double-stranded DNA in the cell after delivery.

In some embodiments, the adverse effect is dose-dependent to the exogenous nucleic acid delivered to the subject.

In some embodiments, the sub-therapeutic amount is no more than 90%, no more than 80%, no more than 70%, no more than 60%, no more than 50%, no more than 40%, no more than 30%, no more than 20%, no more than 10%, no more than 5%, no more than 2%, or no more than 1% of the conventional amount of the same exogenous nucleic acid that would otherwise be required without the administration of the aspirin compound.

In some embodiments, the exogenous nucleic acid comprises an AAV vector, optionally comprising an AAV virus particle. In some embodiments, the sub-therapeutic amount of the AAV vector or AAV virus particle is no more than 107 vg/kg (vector genomes/kg), no more than 10⁸ vg/kg, no more than 10⁹ vg/kg, no more than 10¹⁰ vg/kg, no more than 10¹¹ vg/kg, no more than 10¹² vg/kg, no more than 10¹³ vg/kg, or no more than 10¹⁴ vg/kg.

In some embodiments, the aspirin compound is administered to the subject at an amount of no more than 30 mg/kg, no more than 50 mg/kg, no more than 100 mg/kg, no more than 110 mg/kg, no more than 120 mg/kg, no more than 120 mg/kg, no more than 130 mg/kg, no more than 140 mg/kg, no more than 150 mg/kg, no more than 160 mg/kg, no more than 170 mg/kg, no more than 180 mg/kg, no more than 190 mg/kg, or no more than 200 mg/kg.

In some embodiments, the aspirin compound is administered to the subject at least 1 day, 2 days, 3 days, 4 days, 5 days, 6 days or 7 days prior to the delivery of the exogenous nucleic acid to the subject, and/or is administered for once or repetitively (e.g. twice, three times, four times and so on) prior to the delivery of the nucleic acid. In some embodiments, the aspirin compound and/or the exogenous nucleic acid is administered via parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, or intrathecal administration routes to the subject.

In yet another aspect, the present disclosure provides a pharmaceutical composition comprising a sub-therapeutic amount of an exogenous nucleic acid and a pharmaceutically acceptable carrier, optionally further comprising an aspirin compound, wherein the exogenous nucleic acid comprises double-stranded DNA, or can be converted to double-stranded DNA in a cell after delivery of the exogenous nucleic acid to the cell.

In yet another aspect, the present disclosure provides a pharmaceutical composition comprising an exogenous nucleic acid, an aspirin compound, and a pharmaceutically acceptable carrier, wherein the exogenous nucleic acid comprises double-stranded DNA, or can be converted to double-stranded DNA in a cell after delivery of the exogenous nucleic acid to the cell.

In some embodiments, the pharmaceutical composition of the present disclosure further comprises an instruction for use that indicates the aspirin compound is to be administered prior to or concurrently with administration of the pharmaceutical composition.

In some embodiments, the exogenous nucleic acid comprises an AAV vector, optionally comprising an AAV virus particle. In some embodiments, the pharmaceutical composition is in a unit dose, and contains no more than 10¹⁰ vg, 10^{10.5} vg, 10¹¹ vg, 10^{11.5} vg, 10¹² vg, 10^{12.5} vg, 10¹³ vg, 10^{13.5} vg, 10¹⁴ vg, 10^{14.5} vg, 10¹⁵ vg, 10^{15.5} vg, or 10¹⁶ vg of AAV virus particle.

In a further aspect, the present disclosure provides a kit comprising: a) a first composition comprising an aspirin compound; and b) a second composition comprising an exogenous nucleic acid, wherein the exogenous nucleic acid comprises double-stranded DNA, or can be converted to double-stranded DNA in a cell after delivery of the exogenous nucleic acid to the cell.

In some embodiments, the kit further comprising an instruction for use that indicates that the first composition is to be administered prior to or concurrently with the second composition. In some embodiments, the first composition and the second composition can be readily mixed to provide a combined composition before use. In some embodiments, the second composition comprises the exogenous nucleic acid at a sub-therapeutic amount.

In a further aspect, the present disclosure provides a kit comprising a composition comprising an aspirin compound and an exogenous nucleic acid, wherein the exogenous nucleic acid comprises double-stranded DNA, or can be converted to double-stranded DNA in a cell after delivery of the exogenous nucleic acid to the cell.

In yet a further aspect, the present disclosure provides a composition comprising: an aspirin compound and an exogenous nucleic acid in combination, wherein the exogenous nucleic acid comprises double-stranded DNA, or can be converted to double-stranded DNA in a cell after delivery of the exogenous nucleic acid to the cell.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows AAV-mediated expression of luciferase gene in mice treated with aspirin at different concentrations.

FIG. 2 shows IFN-α levels on 3 days post AAV injection with aspirin treatment at different concentrations.

FIGS. 3A-3C show expression levels of luciferase after injection of AAV8 (FIG. 3A), AAV9 (FIG. 3B) and AAV843 (FIG. 3C) in mice treated with aspirin and in the control group.

FIGS. 4A-4B shows IFN-α (FIG. 4A) and IFN-β (FIG. 4B) levels after injection of AAV8 in mice treated with aspirin and in the control group.

FIGS. 5A-5B shows IFN-α (FIG. 5A) and IFN-β (FIG. 5B) levels after injection of AAV9 in mice treated with aspirin and in the control group.

FIGS. 6A-6B shows IFN-α (FIG. 6A) and IFN-β (FIG. 6B) levels after injection of AAV843 in mice treated with aspirin and in the control group.

FIGS. 7A-7D shows mRNA level (FIG. 7A, 7C) and enzymatic activity level (FIG. 7B, 7D) of Gluc in brain tissue or in liver tissue of mice receiving AAV9-CB-Gluc with pre-injection of aspirin, or simultaneous injection of aspirin, or without aspirin.

FIG. 8 shows the IDS enzyme activity in brain after treatment with AAV9-CB-IDS vector in MPSII mice at 3×10¹³ vg/kg or 1×10¹⁴ vg/kg, or at 3×10¹³ vg/kg in combination with pretreatment of aspirin at 50 mg/kg.

FIG. 9 shows all the sequences disclosed in the present disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub combination. In order to illustrate, if one specific embodiment disclosed herein includes component A, B and C, it should be understood that the present disclosure also intends to include embodiments which contains A, B or C individually or any combinations of A, B or C.

Unless otherwise defined, all technological and scientific terminologies used herein have the identical meaning generally understood by those of skills in the art.

All publications, patent applications, patents, and other references mentioned herein are incorporated by reference herein in their entirety.

As used herein, “a,” “an,” or “the” can mean one or more than one. For example, “a” cell can mean a single cell or a plurality of cells.

As used herein, unless specifically indicated otherwise, the word “or” is used in the inclusive sense of “and/or” and not the exclusive sense of “either/or.”

Unless specifically indicated otherwise, the number range described herein can include each number within the range and each subrange.

The term “about,” as used herein when referring to a measurable value such as an amount of dose, time, temperature, activity or other biological activity and the like, is meant to encompass variations of 20%, ±10%, ±5%, ±1%, ±0.5%, or even ±0.1% of the specified amount.

The present invention is at least partially based on the discovery that administration of an aspirin compound to a cell or a subject prior to or concurrently with the delivery of the exogenous nucleic acid can significantly increase the expression of the exogenous nucleic acid in the cell or in the subject.

In another aspect, the present disclosure provides methods of increasing expression level of, or prolonging expression duration of, an exogenous nucleic acid in a cell or in a subject, the method comprising: administering to the cell or the subject an aspirin compound prior to or concurrently with the delivery of the exogenous nucleic acid to the cell or the subject, wherein the exogenous nucleic acid comprises double-stranded DNA, or can be converted to double-stranded DNA in the cell after delivery, and whereby the expression level or the expression duration of the exogenous nucleic acid is increased or prolonged as compared to a control expression level or a control expression duration, respectively, obtained without the administration of the aspirin compound.

In another aspect, the present disclosure provides methods of expressing, or increasing expression level of, or prolonging expression duration of, an exogenous nucleic acid in a cell or in a subject, the method comprising: delivering to the cell or the subject the exogenous nucleic acid in a condition suitable for expression, wherein the cell or the subject has been or is concurrently being administered with an aspirin compound; and wherein the exogenous nucleic acid comprises double-stranded DNA, or can be converted to double-stranded DNA in the cell or in the subject after delivery.

In another aspect, the present disclosure provides methods of expressing, or increasing expression of, or prolonging expression duration of, an exogenous nucleic acid in a cell or in a subject, the method comprising:

a) administering an aspirin compound to the cell or to the subject; and
b) delivering to the cell or to the subject the exogenous nucleic acid in a condition suitable for expression,
wherein the step a) is prior to or concurrently with the step b), wherein the exogenous nucleic acid comprises double-stranded DNA, or can be converted to double-stranded DNA in the cell or in the subject after delivery.

In one aspect, the present disclosure provides methods of priming a cell for delivery of an exogenous nucleic acid, the method comprising: administering an aspirin compound to the cell prior to or concurrently with the delivery of the exogenous nucleic acid to the cell, wherein the exogenous nucleic acid comprises double-stranded DNA, or can be converted to double-stranded DNA in the cell after delivery.

In another aspect, the present disclosure provides methods of priming a subject having a condition treatable by an exogenous nucleic acid or the expression product thereof, the method comprising: administering to the subject an aspirin compound prior to or concurrently with the delivery of the exogenous nucleic acid to the cell, wherein the exogenous nucleic acid comprises double-stranded DNA, or can be converted to double-stranded DNA in the subject after delivery.

In another aspect, the present disclosure provides methods of treating or preventing a condition treatable or preventable by an exogenous nucleic acid or the expression product thereof, the method comprising: delivering to the subject the exogenous nucleic acid, wherein the exogenous nucleic acid comprises double-stranded DNA, or can be converted to double-stranded DNA in the subject after delivery, and wherein the subject has been or is concurrently being administered with an aspirin compound.

In another aspect, the present disclosure provides methods of treating or preventing a condition treatable or preventable by an exogenous nucleic acid or the expression product thereof, the method comprising: a) administering an aspirin compound to the subject; and b) delivering to the subject the exogenous nucleic acid, wherein the step a) is prior to or concurrently with the step b), wherein the exogenous nucleic acid comprises double-stranded DNA, or can be converted to double-stranded DNA in the subject after delivery.

The term “aspirin compound” as used herein include aspirin, analogs, and derivatives thereof. Aspirin is also known as acetylsalicyclic acid, and has a chemical structure shown below:

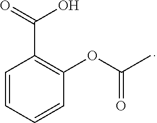

Derivatives of aspirin include, but are not limited to, salts (e.g. pharmaceutically acceptable salts), esters, solvates, and prodrugs of aspirin, which can provide for acetylsalicyclic acid or any of its active form, for example, after processing in vivo by hydrolysis or metabolism. Exemplary salts of aspirin include but are not limited to, Aspirin-arginine (which is the double salt formed by L-arginine and acetylsalicylic acid, also named Arginine Aspirin), lithium acetylsalicylate (e.g. Hydropyrin®), sodium acetylsalicylate (e.g. Catalgine®), calcium acetylsalicylate (e.g. Kalmopyrin®, Ascal®, Dispril®, “Kalsetal®, Solaspin®, Solprin®, Tylcasin®, Alcacyl®, Calurin®, Ironin®, Solupsan®) and magnesium acetylsalicylate (e.g. Novacyl®). Exemplary esters of aspirin include but are not limited to, glycolamide, glycolate, (acyloxy)methyl, alkyl, and aryl esters of acetylsalicylic acid.

Analogs of aspirin are compounds which are functional equivalents of aspirin, but do not have aspirin's chemical structure, and is not aspirin's derivative. Analogs of aspirin can have similar (though not identical) structures as compared to aspirin, and also share the same biological activity of aspirin as provided herein.

### Exogenous Nucleic Acid

As used herein, the term “exogenous nucleic acid” is a nucleic acid that is to be delivered to a cell or a subject. The exogenous nucleic acid may be linear or circular, and can be in the form of a naked nucleic acid or can be in a packaged form such as a virus particle. The exogenous nucleic acid may comprise a sequence which is not naturally found in the cell or the subject. The exogenous nucleic acid comprises double-stranded DNA, or can be converted to double-stranded DNA in a cell after delivery of the exogenous nucleic acid to the cell.

In certain embodiments, the exogenous nucleic acid comprises double-stranded deoxyribonucleic acids (DNA). The exogenous nucleic acid can be composed of double-stranded DNA over its entire length, for example, as a double-stranded DNA (dsDNA) virus vector, a dsDNA virus particle, a double-stranded plasmid, a double-stranded artificial chromosome or exosome, or a double-stranded naked DNA. Alternatively, the exogenous nucleic acid can comprise partially dsDNA, for example, the exogenous nucleic acid can be a single-stranded DNA having secondary double-stranded structures over a partial sequence.

In certain embodiments, the exogenous nucleic acid comprise a nucleic acid that is not dsDNA but can be converted to dsDNA in the cell after delivery. Examples of such nucleic acids include, without limitation, single-stranded DNA (which can be converted to dsDNA by for example DNA polymerase), and RNA (which can be reverse transcribed into dsDNA by reverse transcriptase) such as a retrovirus vector, a retrovirus particle, a lentivirus vector, and a lentivirus particle. In certain embodiments, such nucleic acids can be converted into dsDNA in the cytosol of the cell.

In certain embodiments, the exogenous nucleic acid comprises a vector. The term “vector” means any nucleic acid molecule (whether naked or packaged) for the cloning of and/or transfer of a nucleic acid into a cell. A vector includes both viral and non-viral (e.g., plasmid, exosomes) nucleic acid molecules for introducing a nucleic acid into a cell in vitro, ex vivo, and/or in vivo. In some embodiments, a vector can be recombinant in that it contains one or more heterologous nucleotide sequences such as a transgene or a heterologous regulatory sequences.

In certain embodiments, the exogenous nucleic acid comprises a plasmid. As used herein, the term “plasmid” refers to a construction comprised of extra-chromosomal genetic material, usually of a circular duplex of DNA which can replicate independently of chromosomal DNA. Plasmids are commonly used in gene transfer, as the vehicle by means of which DNA fragments can be introduced into a host organism.

In certain embodiments, the exogenous nucleic acid comprises or is contained within a viral vector. As used herein, an “viral vector” refers to a nucleic acid vector, either single-stranded or double-stranded, having a 5′ viral terminal repeat and/or 3′ viral terminal repeat sequences at the 5′ end and/or 3′ end of a nucleic acid sequence of interest (for example, an expression construct encoding a protein of interest). A viral vector can comprises a pair of TRs or a single TR. The term “terminal repeat” or “TR” includes any viral terminal repeat or synthetic sequence that forms a hairpin structure and mediates the desired functions such as replication, virus packaging, integration and/or provirus rescue, and the like. For example, the 5′ viral terminal repeat and 3′ viral terminal repeat sequences can contain originals of replication, and allow for DNA synthesis initiation at one of the viral terminal repeat and proceeds to the other viral terminal repeat. Examples of viral terminal repeats include but are not limited to, inverted terminal repeats (ITR) (for example, those includes in the AAV), long terminal repeats (LTR) (for example, those included in the retrovirus) etc. The viral vector can comprise, between the ITRs, one or more sequences that are heterologous to the viral genome.

A viral vector as used herein can also encompass virus particles produced from one or more vectors containing viral nucleic acid sequences. A “virus particle” as used herein means a viral genome packaged within a viral capsid. The viral genome in the virus particle can be a modified viral genome such that it may lack some of the native viral sequences and/or may contain some sequences heterologous to the native viral genome.

A variety of viral vectors are known in the art to be suitable for delivering nucleic acids to cells or to a subject such as human. The most commonly used viral vectors include those derived from adenovirus, adeno-associated virus (AAV) and retrovirus, including lentivirus such as human immunodeficiency virus (HV). Retroviral vectors, adenoviruses and AAV offer an efficient, and useful means of introducing and expressing exogenous genes efficiently in mammalian cells. These vectors have broad host and cell type ranges, and express genes stably and efficiently. The safety of these vectors has been understood in the art. Other virus vectors that may be used for gene transfer into a subject include herpes virus papovaviruses such as JC, SV40, polyoma; Epstein-Barr Virus (EBV); papilloma viruses, e.g. bovine papilloma virus type I (BPV); poliovirus and other human and animal viruses.

In certain embodiments, the exogenous nucleic acid comprises an AAV vector. AAV is a single-stranded human DNA parvovirus whose genome has a size of about 4.7 kb. The AAV genome contains two major genes: the rep gene, which codes for the rep proteins (Rep 76, Rep 68, Rep 52 and Rep 40) and the cap gene, which codes for AAV structural proteins (VP-1, VP-2 and VP-3), flanked by 5′ inverted terminal repeat (ITR) and 3′ ITR. The term “AAV vector” as used herein encompasses any viral vector that comprises one or more heterologous sequence flanked by at least one, or two AAV inverted terminal repeat sequences. The term “AAV ITR”, as well-understood in the art, is an approximately 145-nucleotide sequence that is present at both termini of the native single-stranded AAV genome. The outermost 125 nucleotides of the JTR can be present in either of two alternative orientations, leading to heterogeneity between different AAV genomes and between the two ends of a single AAV genome. The outermost 125 nucleotides also contains several shorter regions of self-complementarity, allowing intra-strand base-pairing to occur within this portion of the ITR.

An AAV ITR can be derived from any AAV, including but not limited to AAV serotype 1 (AAV 1), AAV 2, AAV 3, AAV 4, AAV 5, AAV 6, AAV 7, AAV 8, AAV 9, AAV 10, AAV 11, AAV 12, avian AAV, bovine AAV, canine AAV, equine AAV, and ovine AAV and any other AAV now known or later discovered. For details please see, e.g., BERNARD N F et al., VIROLOGY, volume 2, chapter 69 (4th ed., Lippincott-Raven Publishers), Gao et al., (2004) J. Virol. 78:6381-6388. The nucleotide sequences of AAV ITR regions are known. See for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). An early description of the AAV1, AAV2 and AAV3 terminal repeat sequences is provided by Xiao, X., (1996), “Characterization of Adeno-associated virus (AAV) DNA replication and integration,” Ph.D. Dissertation, University of Pittsburgh, Pittsburgh, Pa. (incorporated herein to it its entirety).

An AAV ITR can be native AAV ITR, or alternatively can be altered from a native AAV ITR, for example by mutation, deletion or insertion, so long as the altered ITR can still mediate the desired biological functions such as replication, virus packaging, integration, and the like. The 5′ and 3′ ITRs which flank a selected nucleotide sequence in an AAV vector need not necessarily be identical or derived from the same AAV serotype, so long as they function as intended, for example, to allow for excision and rescue of the sequence of interest from and integration into the recipient cell genome.

The genomic sequence of AAV as well as AAV rep genes, and cap genes are known in the art, can be found in the literature and in public database such as the GenBank database. Table 1 below shows some exemplary sequences for AAV genomes or AAV capsid sequences, and more are reviewed in Bernard N F et al., VIROLOGY, volume 2, chapter 69 (4th ed., Lippincott-Raven Publishers); Gao et al., (2004) J. Virol. 78:6381-6388; Naso M F et al., BioDrugs. 2017; 31(4): 317-334.

TABLE 1

| AAV | GenBank Accession No. |
|---|---|
| AAV1 | NC_002077; AF063497 |
| AAV2 | NC_001401 |
| AAV3 | NC_001729 |
| AAV4 | NC_001829 |
| AAV5 | Y18065, AF085716 |
| AAV6 | NC_001862 |
| AAV7 | NC_006260.1; AF513851 |
| AAV8 | NC_006261.1; AF513852, |
| Avian AAV | NC_004828 |
| ATCC VR-865 | |
| Avian AAV | NC_006263 |
| strain DA-1 | |
| Bovine AAV | NC_005889 |

In some embodiments, the AAV vectors can be recombinant. A recombinant AAV vector can comprise one or more heterologous sequences that is not of the same viral origin (e.g. from a non-AAV virus, or from a different serotype of AAV, or from a partially or completely synthetic sequence). In certain embodiments, the heterologous sequence is flanked by the at least one AAV ITR.

In certain embodiments, the AAV vectors provided herein have a size suitable for being packaged into an AAV virus particle. For example, the size of the AAV vectors can be up to the size limit of the genome size of the AAV to be used, for example, up to 5.2 kb. In certain embodiments, the AAV vector has a size of no more than 5.2 kilobases (kb), no more than about 5 kb, no more than about 4.5 kb, no more than about 4 kb, no more than about 3.5 kb, no more than about 3 kb, no more than about 2.5 kb in size, see for example, Dong, J. Y et al. (Nov. 10, 1996).

Due to the packaging size limit of a single AAV, for a heterologous sequence that exceed the packaging capacity of a single AAV vector, two or more AAV vectors can be constructed in a way that permits reconstitution to a complete sequence or expression cassette in a cell co-transfected with these AAV vectors. Methods are known in the art to construct such AAV vectors, for example to construct overlapping dual vectors, trans-splicing vector-pairs, hybrid vector systems, and more details are available at, Chamberlain K et al, Hum Gene Ther Methods. 2016 Feb. 1; 27(1): 1-12, and U.S. Pat. No. 6,596,535.

AAV vectors can be constructed using methods known in the art. General principles of rAAV vector construction are known in the art. See, e.g., Carter, 1992, Current Opinion in Biotechnology, 3:533-539; and Muzyczka, 1992, Curr Top. Microbiol. Immunol., 158:97-129. For example, a heterologous sequence can be directly inserted between the ITRs of an AAV genome in which the Rep gene and/or Cap gene have been deleted. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

Alternatively, AAV ITRs can be excised from the viral genome or from an AAV vector containing the same, and fused to 5′ and 3′ of a heterologous sequence using standard ligation techniques, such as those described in Sambrook et al., supra. AAV vectors which contain AAV ITRs are commercially available and have been described in, e.g., U.S. Pat. No. 5,139,941.

In certain embodiments, the AAV vector comprises a recombinant AAV virus particle. The AAV virus particle can be produced from an AAV expression vector. AAV particles can be produced by introducing an AAV expression vector into a suitable host cell using known techniques, such as by transfection, together with other necessary machineries such as plasmids encoding AAV cap/rep gene, and helper genes provided by either adeno or herpes viruses (see, for example, M. F. Naso et al, BioDrugs, 31(4): 317-334 (2017), which are incorporated herein to its entirety). The AAV expression vector can be expressed in the host cell and packaged into virus particles.

In some embodiments, the AAV vector further comprises a cap gene which encodes a capsid protein. In some embodiments, the AAV vector comprises an AAV virus particle comprising a native or recombinant capsid protein. In some embodiments, the capsid protein can be modified or chimeric or synthetic. A modified capsid can comprise modifications such as insertions, additions, deletions, or mutations. For example, a modified capsid may incorporate a detection or purification tag. A chimeric capsid comprises portions of two or more capsid sequences. A synthetic capsid comprise synthetic or artificially designed sequence. The capsid structure of AAV is also known in the art and described in more detail in Bernard N F et al., supra. In some embodiments, the cap gene or the capsid protein is derived from two or more AAV serotypes. As used herein, the term “serotype” with respect to an AAV refers to the capsid protein reactivity with defined antisera. It is known in the art that various AAV serotypes are functionally and structurally related, even at the genetic level (see; e.g., Blacklow, pp. 165-174 of “Parvoviruses and Human Disease” J. R. Pattison, ed. (1988); and Rose, Comprehensive Virology 3:1, 1974). However, AAV virus particles of different serotypes may have different tissue tropisms (see, for details, in, Nonnenmacher M et al., Gene Ther., 2012 June; 19(6): 649-658), and can be selected as appropriate for gene therapy for a target tissue. In some embodiments, the cap gene or the capsid protein can have a specific tropism profile. The term “tropism profile” refers to the pattern of transduction of one or more target cells, tissues and/or organs. For example, the capsid protein may have a tropism profile specific for liver (e.g. hepatocytes), brain, eye, muscle, lung, kidney, intestine, pancreas, salivary gland, or synovia, or any other suitable cells, tissue or organs.

In some embodiments, the cap gene or the capsid protein is derived from any suitable AAV capsid gene or protein, for example, without limitation, AAV capsid gene or protein derived from AAV1, AAV2, AAV3, AAV4, AAV5, AAV6, AAV7, AAV8, AAV9, AAV12, AAV843, AAVbb2, AAVcyS, AAVrh10, AAVrh20, AAVrh39, AAVrh43, AAVrh64, AAVhu37, AAV3B, AAVhu48, AAVhu43, AAVhu44, AAVhu46, AAVhu19, AAVhu20, AAVhu23, AAVhu22, AAVhu24, AAVhu21, AAVhu27, AAVhu28, AAVhu29, AAVhu63, AAVhu64, AAVhu13, AAVhu56, AAVhu57, AAVhu49, AAVhu58, AAVhu34, AAVhu45, AAVhu47, AAVhu51, AAVhu52, AAVhu T41, AAVhu 517, AAVhu T88, AAVhu T71, AAVhu T70, AAVhu T40, AAVhu T32, AAVhu T17, AAVhu LG15, AAVhu9, AAVhu10, AAVhu11, AAVhu53, AAVhu55, AAVhu54, AAVhu7, AAVhu18, AAVhu15, AAVhu16, AAVhu25, AAVhu60, AAVch5, AAVhu3, AAVhu1, AAVhu4, AAVhu2, AAVhu61, AAVrh62, AAVrh48, AAVrh54, AAVrh55, AAVcy2, AAVrh35, AAVrh37, AAVrh36, AAVcy6, AAVcy4, AAVcy3, AAVcy5, AAVrh13, AAVrh38, AAVhu66, AAVhu42, AAVhu67, AAVhu40, AAVhu41, AAVrh40, AAVrh2, AAVbb1, AAVhu17, AAVhu6, AAVrh25, AAVpi2, AAVpi3, AAVrh57, AAVrh50, AAVrh49, AAVhu39, AAVrh58, AAVrh61, AAVrh52, AAVrh53, AAVrh51, AAVhu14, AAVhu31, AAVhu32, AAVrh34, AAVrh33, AAVrh32, Avian AAV ATCC VR-865, Avian AAV strain DA-1 or Bovine AAV

The capsid of AAV843 is the identical to the synthetic capsid AAVXL32 as disclosed in WO2019241324A1 (incorporated herein to its entirety), and AAV843 is also disclosed in for example, Xu J. et al., Int J Clin Exp Med, 2019; 12(8):10253-10261. In certain embodiments, the capsid protein of AAV843 has an amino acid sequence of SEQ ID NO: 10. In certain embodiments, the capsid gene encoding capsid protein of AAV843 has a nucleic acid sequence that is at least 90%, 92%, 95%, 97% or 98% identical to SEQ ID NO: 6, or that is a variant of SEQ ID NO: 6 having degenerate codon substitutions. Degenerate codon substitutions, also known as synonymous nucleotide substitution, may be achieved by generating sequences in which the third position of one or more selected (or all) codons is substituted with mixed-base and/or deoxyinosine residues.

In certain embodiments, the capsid protein of AAV8 has an amino acid sequence of SEQ ID NO: 8. In certain embodiments, the capsid gene encoding capsid protein of the capsid protein of AAV8 has a nucleic acid sequence that is at least 90%, or 95% identical to SEQ ID NO: 4, or that is a variant of SEQ ID NO: 4 having degenerate codon substitutions.

In certain embodiments, the capsid protein of AAV9 has an amino acid sequence of SEQ ID NO: 9. In certain embodiments, the capsid gene encoding capsid protein of AAV9 has a nucleic acid sequence that is at least 90%, or 95% identical to SEQ ID NO: 5, or that is a variant of SEQ ID NO: 5 having degenerate codon substitutions.

More examples of AAV capsid gene sequences and protein sequences can be found in GenBank database, see, GenBank Accession Nos: AF043303, AF028705, AF028704, J02275, J01901, J02275, X01457, AF288061, AH009962, AY028226, AY028223, NC 001358, NC 001540, AF513851, AF513852, AY530579, AY631965, AY631966; AF063497, AF085716, AF513852, AY530579, AAS99264.1, AY243022, AY243015, AY530560, AY530600, AY530611, AY530628, AY530553, AY530606, AY530583, AY530555, AY530607, AY530580, AY530569, NC 006263, NC 005889, NC 001862, AY530609, AY530581, AY530563, AY530591, AY530562, AY530584, AY530622, AY530601, AY530586, AY243021, AY530570, AY530589, AY530595, AY530572, AY530588, AY530575, AY530565, AY530590, AY530602, AY530566, AY530587, AY530585, AY530564, AY530592, AY530623, AY530574, AY530593, AY530560, AY530594, AY530573, AF513852, AY530624, AY530561, AY242997, AY530625, AY530567, AY530556, AY530578, AY530568, AY530618, AY243020, AY530579, AY530619, AY530596, AY530612, AY243000, AY530597, AY530620, AY242998, AY530598, AY242999, AY530599, AY243016, NC 001729, NC 001401, AY243018, NC 001863, AY530608, AY243019, NC 001829, AY530610, AY243017, AY243001, AY530613, AY243013, AY243002, AY530614, AY243003, AY695378, AY530558, AY530626, AY695376, AY695375, AY530605, AY695374, AY530603, AY530627, AY695373, AY695372, AY530604, AY695371, AY530600, AY695370, AY530559, AY695377, AY243007, AY243023, AY186198, AY629583, NC 004828, AY530629, AY530576, AY243015, AY388617, AY530577, AY530582, AY530615, AY530621, AY530617, AY530557, AY530616 or AY530554.

In certain embodiments, the AAV vector comprises a cap gene from one AAV serotype and AAV ITRs from a second serotype. In certain embodiments, the AAV vector comprises an AAV virus particle comprising a pseudotyped AAV “Pseudotyped” AAV refers to an AAV that contains capsid proteins from one serotype and a viral genome including 5′-3′ ITRs of a second serotype. Pseudotyped AAV would be expected to have cell surface binding properties of the serotype from which the capsid protein is derived and genetic properties consistent with the serotype from which the ITRs are derived.

In certain embodiments, the exogenous nucleic acid comprises an adenovirus vector. Adenovirus has a double-strand linear DNA genome which cannot be integrated into the host genome. Illustrative examples of adenovirus vectors include, without limitation, first generation adenovectors (e.g., adenovector having E1a and E1b genes deleted, and adenovector having E1 and E3 genes deleted), second generation adenoviral vectors (e.g. adenovector having E1 and E2 genes deleted, adenovector having E1 and E4 genes deleted), and gutless adenoviral vectors in which all viral coding sequences are deleted (also called helper dependent adenoviral vector).

In certain embodiments, the exogenous nucleic acid comprises a retrovirus vector. A retrovirus has a RNA genome and can replicate in a host cell via reverse transcriptase to produce DNA from the RNA genome. Illustrative examples of retrovirus vectors include, without limitation, vectors derived from avian leucosis virus, mouse mammary tumor virus, murine leukemia virus, bovine leukemia virus, Walleye dermal sarcoma virus, HIV-1 (Human Immunodeficiency Virus), HIV-2, SIV (simian immunodeficiency virus), EIAV (equine infectious anaemia virus), FIV (feline immunodeficiency virus), CAEV (caprine arthritis encephalitis virus), VMV (visna/maedi virus), human spumavirus, moloney murine leukaemia virus, rous sarcoma virus, feline leukemia virus, human T-lymphotropic virus, and simian foamy virus.

In certain embodiments, the exogenous nucleic acid comprises a lentivirus vector. Lentiviruses are complex retroviruses which, in addition to the common retroviral genes gag, pol and env, contain other genes with regulatory or structural function. Illustrative examples of lentivirus vectors include, without limitation, vectors derived from HIV-1, SIV, FIV, CAEV, VMV, and EIAV

In certain embodiments, the exogenous nucleic acid can comprise an encoding sequence that encodes for a protein of interest, or a portion thereof. In some embodiments, the encoding sequence for the protein of interest can be divided or split into two or more exogenous nucleic acid sequences (for example, two or more plasmids, or two or more viral particles), in a way to allow the divided encoding sequences to be joined together after delivery into the cell, for example, by homologous recombination, or via certain viral packaging process. This would permit encoding sequences whose length exceeds the delivery capacity of the vector (such as AAV vector or plasmid) to be delivered and expressed.

The protein of interest can be any protein whose expression in the cell or in the subject is of interest. In certain embodiments, the protein of interest can be a therapeutic protein (e.g. for medical or veterinary uses), immunogenic protein (e.g. for vaccines), a reporter protein, a nuclease or a therapeutic target protein.

Therapeutic proteins can be expressed in vitro to provide for a therapeutic composition to be delivered to a subject in need thereof, or can be expressed in vivo to provide for therapeutic benefit. Examples of such therapeutic proteins include, without limitation, an antibody (e.g. monoclonal or bispecific or multi-specific), insulin, glucagon-like peptide-1, peptide hormones, growth factors, erythropoietin (EPO), cytokines, coagulation factors, antihemophilic factors, interferons, Fc-fusion proteins (such as CTLA-4 Fc-fusion, VEGFR Fc-fusion), therapeutic enzymes (e.g. lysosomal hydrolase, and sulfatases). Alternatively, therapeutic proteins can be expressed in vivo in a subject in need thereof. Examples of such therapeutic proteins include but are not limited to survival of motor neuron 1 (SMN1, gene ID: 6066), alpha-N-acetylglucosaminidase (NAGLU, gene ID:4669), N-sulphoglucosamine sulphohydrolase (SGSH, gene ID: 6448), iduronate 2-sulfatase (IDS, gene ID:3423), coagulation factor VIII (FVIII, gene ID:2157), coagulation factor IX (FIX, gene ID: 2158), Bruton tyrosine kinase (BTK, gene ID:695), ATP binding cassette subfamily D member 1 (ABCD1, gene ID:215), acyl-CoA dehydrogenase very long chain (ACADVL, gene ID: 37), androgen receptor repeat instability region (AR, gene ID: 109504725), hemoglobin subunit beta (HBB, gene ID: 3043), sodium voltage-gated channel alpha subunit 1 (SCN1A, gene ID: 6323), CF transmembrane conductance regulator (CFTR, gene ID: 1080), colony stimulating factor 2 receptor subunit alpha (CSF2RA, gene ID: 1438), interleukin 2 receptor subunit alpha (IL2AG, gene ID: 3559), phenylalanine hydroxylase (PHA, gene ID: 5053), serine/threonine kinase 11 (STK11, gene ID: 6794), phosphatidylinositol glycan anchor biosynthesis class A (PIGA, gene ID: 5277), ornithine carbamoyltransferase (OTC, gene ID: 5009), N-acetylglutamate synthase deficiency (NAGS, gene ID: 162417), DM1 protein kinase (DMPK, gene ID: 1760), CCHC-type zinc finger nucleic acid binding protein (CNBP, gene ID: 7555), acyl-CoA dehydrogenase medium chain (ACADM, gene ID: 34), GNAS complex locus (GNAS, gene ID: 2778), fibrillin 1 (FBN1, gene ID: 2200), lipase A, lysosomal acid type (LIPA, gene ID: 3988), solute carrier family 7 member 7 (SLC7A7, gene ID: 9056), hydroxyacyl-CoA dehydrogenase trifunctional multienzyme complex subunit alpha (HADHA, gene ID: 3030), growth hormone receptor (GHR, gene ID: 2690), isovaleryl-CoA dehydrogenase (IDV, gene ID: 3712), alkaline phosphatase, biomineralization associated (ALPL, gene ID: 249), solute carrier family 25 member 15 (SLC25A15, gene ID: 10166), huntingtin (HTT, gene ID: 3064), holocarboxylase synthetase (HCS, gene ID: 3141), notch receptor 3 (NOTCH3, gene ID: 4854), aldolase, fructose-bisphosphate B (ALDOB, gene ID: 229), ATPase copper transporting beta (ATP7B, gene ID: 540), glucosidase alpha, acid (GAA, gene ID: 2548), glutaryl-CoA dehydrogenase (GCDH, gene ID: 2639), solute carrier family 12 member 3 (SLC12A3, gene ID: 6559), glucosylceramidase beta, (GBA, gene ID: 2629), Familial Mediterranean Fever (MEFV, gene ID: 4210), galactosidase alpha (GLA, gene ID: 2717), chloride voltage-gated channel 1 (CLCN1, gene ID: 1180), nuclear receptor subfamily 0 group B member 1 (NR0B1, gene ID: 190), argininosuccinate synthase 1 (ASS1, gene ID: 445), solute carrier family 25 member 13 (SLC25A13, gene ID: 10165), solute carrier family 22 member 5 (SLC22A5, gene ID: 84), sodium voltage-gated channel alpha subunit 5 (SCN5A, gene ID: 6331), biotinidase (BTD, gene ID: 686), acetyl-CoA acetyltransferase 1 (ACAT1, gene ID: 38), arginase (ARG1, gene ID: 383), cytochrome P450 family 21 subfamily A member 2 (CYP21A2, gene ID: 1589). Still in another embodiment, the therapeutic protein can be expressed ex vivo for example on a T cell to be transplanted to the subject. Such therapeutic protein include for example, a chimeric antigen receptor (CAR).

In certain embodiments, the protein of interest can be an immunogenic protein. An immunogenic protein, or immunogen, may be any polypeptide suitable for protecting the subject against a disease, including but not limited to infectious diseases such as microbial, bacterial, protozoal, parasitic, fungal and viral diseases, and cancer. For example, the immunogen may be an orthomyxovirus immunogen (e.g., an influenza virus immunogen, such as the influenza virus hemagglutinin (HA) surface protein or the influenza virus nucleoprotein gene, or an equine influenza virus immunogen), or a lentivirus immunogen (e.g, an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SIV matrix/capsid proteins, and the HIV or SIV gag, pol and env genes products). The immunogen may also be an arenavirus immunogen (e.g., Lassa fever virus immunogen, such as the Lassa fever virus nucleocapsid protein gene and the Lassa fever envelope glycoprotein gene), a poxvirus immunogen (e.g., vaccinia, such as the vaccinia L1 or L8 genes), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP genes), a bunyavirus immunogen (e.g., RVFV, CCHF, and SFS viruses), or a coronavirus immunogen (e.g., an infectious human coronavirus immunogen, such as the human coronavirus envelope glycoprotein gene, or a porcine transmissible gastroenteritis virus immunogen, or an avian infectious bronchitis virus immunogen, or a severe acute respiratory syndrome (SARS) immunogen such as a S [S1 or S2], M, E, or N protein or an immunogenic fragment thereof, or a COVID-19 immunogen). The immunogen may further be a polio immunogen, herpes immunogen (e.g., CMV, EBV, HSV immunogens), mumps immunogen, measles immunogen, rubella immunogen, diphtheria toxin or other diphtheria immunogen, pertussis antigen, hepatitis (e.g., hepatitis A, hepatitis B or hepatitis C) immunogen, or any other vaccine immunogen known in the art. For another example the immunogen may be a tumor or cancer antigen expressed on the surface of a tumor or cancer cell. Exemplary tumor or cancer antigen include, without limitation, b-catenin, BRCA1 gene product, BRCA2 gene product, EpCAM, EGFR, Her2, VEGFR, CD19, PSMA, and so on.

In certain embodiments, the protein of interest can be a reporter protein. A reporter protein can be expressed in a cell to provide for an engineered cell for a biological assay. Examples of reporter proteins include, without limitation, a fluorescent protein (e.g., EGFP, GFP, RFP, BFP, YFP, or dsRED2), an enzyme that produces a detectable product, such as luciferase (e.g., from Gaussia, Renilla, or Pho firms), b-galactosidase, b-glucuronidase, alkaline phosphatase, and chloramphenicol acetyltransferase gene, or proteins that can be directly detected. Virtually any protein can be directly detected by using, for example, specific antibodies to the protein. Additional markers (and associated antibiotics) that are suitable for either positive or negative selection of eukaryotic cells are disclosed in Sambrook and Russell (2001), Molecular Cloning, 3rd Ed., Cold Spring Harbor Laboratory Press, Cold Spring Harbor, N.Y, and Ausubel et al. (1992), Current Protocols in Molecular Biology, John Wiley & Sons, including periodic updates.

In certain embodiments, the protein of interest can be a nuclease. The term “nuclease” as used herein refers to an enzyme which is capable of cleaving a phosphodiester bond within a polynucleotide chain. The nuclease provided herein can be either naturally-occurring or modified. Examples of nuclease useful in the present disclosure include, without limitation, a Zinc-finger nucleases (ZFN), a transcription activator-like effector nucleases (TALEN), or a Cas family protein (such as Cas1, Cas1B, Cas2, Cas3, Cas4, Cas5, Cas6, Cas7, Cas8, Cas9 (also known as Csn1 and Csx12), Cas 10, Cas 11, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologues thereof, or modified versions thereof). Such nuclease can be useful in, for example, genetic engineering or gene editing in a host genome.

In certain embodiments, the protein of interest can be a therapeutic target protein. Examples of a therapeutic target protein, include but are not limited to, a GPCR, CTLA-4, HER2, Nectin-4, Sclerostin, P-Selectin, VEGF, RSVF, VEGFR2, CD79, IL23p19, vWF, IFN-γ, C5, PD-1, PD-L1, CGRP, CD3, CD11a, CD20, CD22, CD30, CD33, CD38, CD40, CD52, IgE, KLK, CCR4, FGF-23, IL-6R, IL-5, IL-23p19, IL-2R, IL-17R, IL-17, CD4, FIX/FX, IL-12, IL-23, IL-1β, IL-5R, IL-6R, IL-4/IL-13, PDGF-α, Dabigatran, SLAMF7, EGFR, PCSK9, GD2, CD3, CD19, α4β7 integrin, α4β1 integrin, PA, BLyS, RANK, TNF-α, EpCAM, GGTA1, Endostatin, and Angiostatin. Expression of a therapeutic target protein in a recombinant cell can be useful in, for example, producing a recombinant cell line for a biological assay, or for screening or identification of a potential therapeutic agent capable of targeting or interacting with the therapeutic target protein.

Alternatively, in certain embodiments, the exogenous nucleic acid can comprise an encoding sequence that encodes for a functional RNA. A functional RNA may be an untranslated RNA acting on a biological target nucleic acid sequence and modulate the target, for example, inhibiting or reducing the expression or activity of the target. For example, a functional RNA can be an antisense oligonucleotide, a ribozyme (e.g., as described in U.S. Pat. No. 5,877,022), RNAs that effect spliceosome-mediated/raw-splicing (see, Puttaraju et al, (1999) Nature Biotech. 17:246; U.S. Pat. Nos. 6,013,487; 6,083,702), interfering RNAs (RNAi) including small interfering RNAs (siRNA) that mediate gene silencing (see, Sharp et al, (2000) Science 287:2431), microRNA, or other non-translated functional RNAs, such as guide RNAs (Gorman et al., (1998) Proc. Nat. Acad. Sci. USA 95:4929; U.S. Pat. No. 5,869,248 to Yuan et al.,), single guide RNAs used in CRISPR technology (see, e.g. U.S. Pat. Nos. 10,266,850, 8,697,359, US20160298134, Adli M et al, Nat Commun 9, 1911 (2018)), and the like. Potential biological target for the functional RNA can include, without limitation, multiple drug resistance (MDR) protein target, a tumor target (e.g. VEGF, Her2, EGFR, PD-L1 and so on), a pathogen target such as a viral surface antigen (e.g. hepatitis B surface antigen gene), a defective gene product (mutated dystrophins), or a therapeutic target as disclosed herein (e.g. myostatin).

The encoding sequence that encodes the protein of interest or that encodes for a functional RNA can be operably linked to one or more regulatory sequences in the exogenous nucleic acid. The term “operably linked” as used herein means that the encoding sequence is directly or indirectly linked to or associated with one or more regulatory sequences in the exogenous nucleic acid, in a manner that allows expression of the protein of interest from the encoding sequence in the cell. The encoding sequence together with the regulatory sequences can be referred to herein as an expression cassette. In certain embodiments, the exogenous nucleic acid can be in the form of an expression vector. Example of transcription regulatory elements include, one or more promoters and/or enhancers and, optionally, a polyadenylation sequence and/or one or more introns inserted between exons of the protein-coding sequence.

The term “regulatory sequence” as used herein refers to any nucleotide sequence that is necessary or advantageous for the expression of an encoding sequence. A regulatory sequence may include, but is not limited to, one or more promoters, enhancers, transcription terminators, polyadenylation sequences, internal ribosome entry sites, and/or one or more introns inserted between exons of the protein-coding sequence.

The term “promoter” as used herein refers to a polynucleotide sequence that can control transcription of an encoding sequence. The promoter sequence includes specific sequences that are sufficient for RNA polymerase recognition, binding and transcription initiation. In addition, the promoter sequence may include sequences that modulate this recognition, binding and transcription initiation activity of RNA polymerases. The promoter may affect the transcription of a gene located on the same nucleic acid molecule as itself or a gene located on a different nucleic acid molecule as itself. Functions of the promoter sequences, depending upon the nature of the regulation, may be constitutive or inducible by a stimulus. A “constitutive” promoter refers to a promoter that functions to continually activate gene expression in host cells. An “inducible” promoter refers to a promoter that activates gene expression in host cells in the presence of certain stimulus or stimuli. In some embodiments, the promoter is a tissue-specific promoter, or a cell-specific promoter. The term “tissue specific promoter” as used herein refers to a promoter that functions to activate gene expression preferentially or exclusively in certain tissue, and has no activity or reduced activity in other tissues. In one embodiment, the promoter is a CNS-specific promoter. Examples of CNS-specific promoters include those isolated from the genes from myelin basic protein (MBP), glial fibrillary acid protein (GFAP), and neuron specific enolase (NSE), synapsin (SYN). Liver-specific promoters include but are not limited to thyroxine-binding globulin (TBG), Apolipoprotein E (APOE), albumin (ALB), α-1 antitrypsin (hAAT). Muscle-specific promoters include but are not limited to Unc-45 Myosin Chaperone B (UNC45B), RIEG/PITX homeobox 3 (PITX3).

Examples of suitable promoters include, but are not limited to, a pol II promoter such as CMV (e.g. the CMV immediate early promoter (CMV promoter)), Chicken β-actin promoter, pol III promoters, adenovirus major late promoter (Ad MLP); a herpes simplex virus (HSV) promoter, epstein barr virus (EBV) promoter, human immunodeficiency virus (HIV) promoter (e.g. the HIV long terminal repeat (LTR) promoter), moloney virus promoter, mouse mammary tumor virus (MMTV) promoter, mouse mammary tumor virus LTR promoter, rous sarcoma virus (RSV) promoter, SV40 early promoter, promoters from human genes such as human myosin promoter, human hemoglobin promoter, human synapsin promoter, human muscle creatine promoter, human metalothionein beta-actin promoter, human ubiquitin C promoter (UBC), mouse phosphoglycerate kinase 1 promoter (PGK), human thymidine kinase promoter (TK), human elongation factor 1 alpha promoter (EF1A), cauliflower mosaic virus (CaMV) 35S promoter, E2F-1 promoter (promoter of E2F1 transcription factor 1), promoter of alpha-fetoprotein, promoter of cholecystokinin, promoter of carcinoembryonic antigen, promoter of C-erbB2/neu oncogene, promoter of cyclooxygenase, promoter of CXC-Chemokine receptor 4 (CXCR4), promoter of human epididymis protein 4 (HE4), promoter of hexokinase type II, promoter of L-plastin, promoter of mucin-like glycoprotein (MUC1), promoter of prostate specific antigen (PSA), promoter of survivin, promoter of tyrosinase related protein (TRP1), and promoter of tyrosinase, synthetic promoters, hybrid promoters, and the like. Such promoter sequences are commercially available from, e.g., Stratagene (San Diego, Calif.).

The term “enhancer” as used herein refers to a nucleotide sequence that increases the transcription and/or translation of the encoding sequence. The enhancer may be operably linked to the 5′ terminus or 3′ terminus of an encoding sequence. Any enhancer that is functional in eukaryotic cells may be used in the present disclosure. Illustrative examples of enhancers include, without limitation, the simian virus 40 (SV40) early gene enhancer, the enhancer derived from the long terminal repeat (LTR) of Rous Sarcoma virus, and the enhancer derived from human cytomegalovirus (CMV).

The term “transcription terminator” as used herein refers to a nucleotide sequence recognized by a eukaryotic cell RNA polymerase to terminate transcription. The terminator sequence may be operably linked to the 3′ terminus of an encoding sequence. In certain embodiments, the terminator may comprise a signal for the cleavage of the RNA such that a polyadenylation site on the RNA can be exposed. Any terminator sequence that is functional in eukaryotic cells may be used in the present disclosure. Illustrative examples of terminator sequences include, without limitation, termination sequences derived from a virus such as the SV40 terminator, and termination sequences derived from known genes such as the bovine growth hormone terminator sequence.

The term “polyadenylation sequence” as used herein refers to a nucleotide sequence which, when transcribed, is recognized by eukaryotic cells as a signal to add polyadenosine residues to the transcribed mRNA. The polyadenylation sequence may be operably linked to the 3′ terminus of an encoding sequence. Any polyadenylation sequence which is functional in eukaryotic cells may be used in the present disclosure. Illustrative examples of polyadenylation sequences include, without limitation, AAUAAA, and SV40 polyadenylation signal.

In certain embodiments, the exogenous nucleic acid can comprises two sequences encoding for two proteins of interest. In such embodiments, the two encoding sequences can be separated by internal ribosome entry site (IRES), which allows translation initiation from the middle of an mRNA sequence, and consequently separate translation of the two or more encoded products. IRES may be operably linked at a position after the 3′ terminus of a first encoding sequence and before the 5′ terminus of a second encoding sequence. Any IRES sequence which is functional in eukaryotic cells may be used in the present disclosure. Illustrative examples of IRES may include, without limitation, picornavirus IRES, pestivirus IRES, aphthovirus IRES, hepatitis A IRES, and hepatitis C IRES.

### Delivery of the Exogenous Nucleic Acid

The cells to be delivered with the exogenous nucleic acid can be in vitro, ex vivo, or in vivo. In certain embodiments, the cells are in vitro, for example, cells adapted or engineered to be suitable for in vitro culturing, such as cells of an established cell line. In certain embodiments, the cells are ex vivo, for example, primary cells isolated or derived from a subject, such as T cells. In certain embodiments, the cells are in vivo cells in a living subject.

The subject to be delivered with the exogenous nucleic acid can be a non-human animal or human. In some embodiments, the subject is a warm blooded mammal, for example, primates, dogs, cats, cows, horse, sheep, goat, rabbits, rats, and mice. In some embodiments, the subject is a primate, for example, a human.

The exogenous nucleic acid can be delivered to the cell or the subject, using a variety of techniques are available for such delivery. To deliver to cells in vitro or ex vivo, the exogenous nucleic acid can be transfected to the cells by calcium chloride-, lithium chloride-, lithium acetate/polyethylene glycol-, calcium phosphate-, DEAE-dextran-, liposome-mediated transfection (Graham et al. (1973) Virol. 52:456-467; Mannino et al. (1988) BioTechniques 6:682-690; Felgner et al. (1987) Proc. Natl. Acad. Sci. USA 84:7413-7417), electroporation (Shigekawa et al. (1988) BioTechniques 6:742-751), direct microinjection (Capecchi, M. R. (1980) Cell 22:479-488), or by using high-velocity tungsten microprojectiles (Klein et al. (1987) Nature 327:70-73). When the exogenous nucleic acid is in the form of a viral vector, it can be transfected to the cells by viral infection. Transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986); Basic Methods in Molecular Biology, Elsevier, and Chu et al. (1981) Gene 13:197.

To deliver to cells in vivo or to a subject, the exogenous nucleic acid can be administered to the subject systemically or in a local treatment area. In certain embodiments, the exogenous nucleic acid is delivered to the subject via parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, or intrathecal administration routes. In certain embodiments, the exogenous nucleic acid is delivered to the subject orally, intravenously, intraperitoneally, intramuscularly, subcutaneously, intrathecally, intra-cerebroventricularly (ICV), or intrarectally.

### Administration of the Aspirin Compound

Prior to or concurrently with the delivery of the exogenous nucleic acid to the cell or to the subject, the aspirin compound can be administered to the cell or to the subject.

In certain embodiments, the exogenous nucleic acid is delivered to a cell or a subject that has been or is concurrently being administered with an aspirin compound.

In certain embodiment, the aspirin compound is administered prior to the delivery of the exogenous nucleic acid, in such a way to prime the cell or the subject for such delivery. In some embodiments, the aspirin compound is administered to the cell or to the subject at least 1 hour, 2 hours, 3 hours, 4 hours, 5 hours, 6 hours, 7 hours, 8 hours, 9 hours, 10 hours, 12 hours, 18 hours, 24 hours, 1.5 days, 2 days, 3 days, 4 days, 5 days, 6 days or 7 days prior to the delivery of the exogenous nucleic acid. In some embodiments, the aspirin compound is administered to the cell or to the subject for once or repetitively (e.g. twice, three times, four times and so on) prior to the delivery of the nucleic acid.

In some embodiments, the aspirin compound is administered to the cell or to the subject concurrently with the exogenous nucleic acid. The term “concurrently” as used herein refers to the arrangement that would lead to co-delivery of the exogenous nucleic acid and the aspirin compound to the cell or to the subject (either in vitro, ex vivo or in vivo in the subject). Such co-delivery can be achieved by for example, delivering both the exogenous nucleic acid and the aspirin compound in a combined composition, or in separate compositions but via the same or different administration routes at substantially the same time, or in separate compositions at a controlled timing such that both the exogenous nucleic acid and the aspirin compound are expected to take effect in the cell or in the subject at substantially the same time.

In some embodiments, the aspirin compound is administered at an amount sufficient to effectively increase expression of the exogenous nucleic acid in the cell or in the subject. In certain embodiments, the aspirin compound is administered at an effective amount to provide for at least 50%, 60%, 70%, 80%, 90%, 100%, 150%, 200%, 250%, 300% or more increase in expression of the exogenous nucleic acid in the cell or in the subject.

In certain embodiments, the aspirin compound can be added or introduced to the culture media of the cells in vitro or ex vivo. In certain embodiments, the aspirin compound is delivered to the subject via parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, or intrathecal administration routes. In certain embodiments, the aspirin compound can be administered to the subject by any suitable routes, for example, without limitation, orally, intravenously, intraperitoneally, intramuscularly, subcutaneously, intrathecally, intra-cerebroventricularly (ICV), or intrarectally. In certain embodiments, the aspirin compound is delivered to the subject by intravenous administration.

### Methods of Priming

In one aspect, the present disclosure provides a method of priming a cell or a subject for delivery of an exogenous nucleic acid.

As used herein, the term “priming” refers to any pre-treatment to cells or to a subject before delivery of the exogenous nucleic acid, so as to prepare the cells or the subject in a status prone to accepting and expressing the exogenous nucleic acid. In certain embodiments, compared with cells or subjects without priming, the primed cell or the primed subjects can be in a status desirable for receiving and expressing exogenous nucleic acid, for example less immunologically responsive to an exogenous nucleic acid.

### Methods of Expressing or Increasing Expression Level or Prolonging Expression Duration

In another aspect, the present disclosure provides a method of expressing an exogenous nucleic acid in a cell. In another aspect, the present disclosure also provides a method of increasing expression level of an exogenous nucleic acid in a cell or in a subject. In yet another aspect, the present disclosure provides a method of prolonging expression duration of an exogenous nucleic acid in a cell or in a subject.

As used herein, the term “expression” or “expressing” refers to transcription of an encoding DNA sequence into mRNA and/or translation of the encoding DNA sequence into a peptide or protein. As used herein, the phrase “expression of the exogenous nucleic acid” refers to expression of an encoding sequence (e.g. a sequence encoding a protein of interest) contained in the exogenous nucleic acid. In some embodiments, the expression level of the exogenous nucleic acid is determined based on mRNA level or protein level. In some embodiments, the expression level is increased by at least 10%, 15%, 20%, 25%, 30%, 35%, 40%, 45%, 50%, 60%, 70%, 80%, 90%, 100%, 120%, 150%, 180%, 200%, 220%, 250%, 280%, 300%, 400%, 500%, 600%, 700%, 800%, or 900%, relative to the control expression level. Control expression level can be an expression level determined at comparable conditions in the absence of treatment with an aspirin compound.

In certain embodiments, the expression level of the exogenous nucleic acid is determined at the 3^{rd} day, 4^{th} day, 5^{th} day, 6^{th} day, 7^{th} day, 8^{th} day, 9^{th} day, 10^{th} day, 11^{th} day, 12^{th} day, 13^{th} day, 14^{th} day, or 15^{th} day after the delivery of the exogenous nucleic acid to the cell or to the subject.

In certain embodiments, the expression level of the exogenous nucleic acid is determined collectively over the period during the expression duration.

As used herein, the “expression duration” is the period during which the exogenous nucleic acid is expressed in the cell or in the subject at a detectable level or at a physiologically or therapeutically effective level. In certain embodiments, the expression duration is the period during which the protein of interest expressed from the exogenous nucleic acid is at or above a detectable or at a physiologically or therapeutically effective level in the cell or in the subject.

In some embodiments of the methods provided herein, the expression duration is prolonged by at least 1 day, 2 days, 3 days, 4 days, 5 days, 6 days, 7 days, 8 days, 9 days or 10 days, relative to a control duration. Control duration can be expression duration determined at comparable conditions in the absence of treatment with an aspirin compound.

### Methods of Treatment or Prevention

In another aspect, the present disclosure provides a method of treating a condition treatable by an exogenous nucleic acid or the expression product thereof.

In some embodiments, the subject is suffering from a condition treatable by an exogenous nucleic acid or the expression product thereof. In some embodiments, the subject is in need of treatment (e.g., the subject would benefit biologically or medically from treatment).

As used herein, a “condition treatable by an exogenous nucleic acid or the expression product thereof” refers to any disease or condition that is susceptible to treatment with an exogenous nucleic acid or the expression product thereof.

In some embodiments, such condition is characterized in deficiency of one or more functional genes or functional protein(s). In some embodiments, such condition is suitable for gene therapy. The exogenous nucleic acid delivered to the subject can be useful to replace or repair the missing or dysfunctional molecular element (e.g. a gene) in the DNA of the living cells in the subject, or alternatively provide for or augment function of the missing or dysfunctional gene in the cell by introducing and expressing a functional gene in the cell.

In some embodiments, the condition is a single gene disorder. A single gene disorder is a disorder caused by one or more abnormalities in the genome that affect one or both copies of a single gene. The genomic abnormality disrupts the gene and leads to missing or insufficient activity of the endogenous protein encoded by the disrupted gene. The symptoms of single gene disorder are resulted from the missing or insufficient activity of the endogenous protein.

In some embodiments, the single gene disorder is an autosomal dominant, autosomal recessive, X-linked, Y-linked or mitochondrial.

Examples of autosomal dominant single gene disorder includes, but are not limited to, Brugada Syndrome, Myotonic Dystrophy 1, Myotonic Dystrophy 2, Hereditary Multy-infarct Dementia, Huntington's disease, neurofibromatosis type 1, neurofibromatosis type 2, Marfan syndrome, Familial hypercholesterolemia (FH), Polycystic kidney disease, Hereditary spherocytosis, hereditary nonpolyposis colorectal cancer, hereditary multiple exostoses, Tuberous sclerosis, Von Willebrand disease, and acute intermittent porphyria), Dravet Syndrome, Peutz-Jeghers Syndrome, achondroplasia, Primary combined immune deficiency, Familial Adenomatous Polyposis (FAP), Spinocerebellar Ataxia, Multiple Endocrine Neoplasia.

Examples of autosomal recessive single gene disorder includes, but are not limited to, Beta-Ketothiolase deficiency, Biotinidase Deficiency, Hepatolenticular Degeneration, Spinal Muscular Atrophy, N-acetylglutamate synthase deficiency, Lysosomal Acid Lipase Deficiency, Lysinuric Protein Intolerance, Long Chain 3-hydroxyacyl-CoA Dehydrogenase Deficiency, Laron syndrome, Isovaleric Acidemia, Hyperornithinemia-hyperammonemia-homocitrullinuria syndrome, Holocarboxylase Synthetase Deficiency, Hereditary Fructose Intolerance, Glycogen storage disease type II, Glutaric Acidemia Type I, Gitelman Syndrome, Gaucher Disease, Familial Mediterranean Fever, Myotonia Congenita, Citrullinemia I, Citrullinemia II, Primary Camitine Deficiency, Arginase Deficiency, Medium-chain acyl-CoA dehydrogenase deficiency, Sickle cell anaemia, cystic fibrosis, Tay-Sachs disease, Phenylketonuria, Lysosomal acid lipase deficiency, Glycogen storage diseases, Galactosemia, Niemann-Pick disease, spinal muscular atrophy (SMA), Roberts syndrome, Very long-chain acyl-CoA dehydrogenase deficiency, Pulmonary Cystic Fibrosis, Gaucher Disease, Werner Syndrome, Fanconi Anemia, Mucopolysaccharidosis (I, IIIA, IIIB, IVA, IVB, VI, VII, IX).

Examples of X-linked single gene disorder includes, but are not limited to, Fragile X syndrome, Congenital Adrenal Hypoplasia, Duchenne muscular dystrophy, and Hemophilia A, Hemophilia B, Fabry Disease, X-linked agammaglobulinemia, X-linked Adrenoleukodystrophy, Spinal and bulbar muscular atrophy, Ornithine Transcarbamylase Deficiency, and Mucopolysaccharidosis II, Adrenoleukodystrophy (ALD), Chronic granulomatous disease.

Other examples of single gene disorder include, McCune-Albright syndrome, Paroxysmal Nocturnal Hemoglobinuria, ADA Imune deficiency, Amyotrophic lateral sclerosis (ALS), glucose-galactose, muscular dystrophy, Azoospermia, Ehlers-Danlos Syndrome, Retinitis Pigmentosa, Hemochromatosis, Melanoma, Retinoblastoma, Alzheimer Disease, Amyloidosis, Myotonic Dystrophy, giant axonal neuropathy, Alpha-1 antitrypsin, Parkinson's disease, Severe Combined Immunodeficiency (ADA-SCID/X-SCID).

Examples of polygenic disorders include, such as Heart disease, Cancer (e.g. leukemia, particularly, acute lymphoblastic leukemia), Diabetes, Schizophrenia and Alzheimer's disease.

As used herein, the term “treating” or “treatment” of a condition as used herein includes alleviating a condition, slowing the onset or rate of development of a condition, reducing, alleviating or ending symptoms associated with a condition, generating a complete or partial regression of a condition, curing a condition, or some combination thereof.

In another aspect, the present disclosure provides a method of preventing a condition preventable by an exogenous nucleic acid or the expression product thereof.

As used herein, the term “prevent,” “preventable,” “prevents,” or “prevention” refers to a delay in the onset of a disease or disorder, reduction in the risk of developing a condition, delaying the development of symptoms associated with a condition, or some combination thereof. The terms are not meant to imply complete abolition of disease and encompasses any type of prophylactic treatment that reduces the incidence of the condition or delays the onset and/or progression of the condition.

In some embodiments, the preventable condition is characterized in a condition that can benefit from a protective effects (e.g. immune response) that can be induced by the exogenous nucleic acid or the expression product thereof. In some embodiments, the exogenous nucleic acid delivered to the subject can be useful to express an immunogen that induces a protective immune response against a pathogen or a cancer cell.

In certain embodiments, the treatment or prevention method comprises: delivering to the subject the exogenous nucleic acid, wherein the subject has been or is concurrently being administered with an aspirin compound.

In certain embodiments, the treatment or prevention method comprises: administering to the subject an aspirin compound prior to or concurrently with the delivery of the exogenous nucleic acid to the cell.

In certain embodiments, the treatment or prevention method comprises: a) administering an aspirin compound to the subject; and b) delivering to the subject the exogenous nucleic acid, wherein the step a) is prior to or concurrently with the step b).

The exogenous nucleic acid is delivered to the subject at a therapeutically effective amount. The term “therapeutically effective amount” as used herein with respect to the exogenous nucleic acid, means that the amount of the exogenous nucleic acid delivered to the subject is sufficient to produce a therapeutic or preventative benefit in the subject, for example, to provide some alleviation, mitigation, or decrease in at least one clinical symptom in the subject (for therapeutic purpose), or to delay the onset of a disease or disorder or the lessening of symptoms upon onset of the disease or disorder (for preventative purpose). For example, a therapeutically effective amount of the exogenous nucleic acid can allow delivery into a sufficient number of the cells and expression of the exogenous nucleic acid (for example, expression of the protein of interest from the exogenous nucleic acid) in the subject to produce a therapeutically benefit or preventative benefit.

In some embodiments, the exogenous nucleic acid comprises or is contained within a viral vector (e.g. AAV vector or AAV virus particle), and the therapeutically effective amount of the viral vector can range from 10⁶ to 10¹⁴ vg/kg (vector genomes/kg), for example, 107 to 10¹⁴ vg/kg, 10¹ to 10¹⁴ vg/kg, 10⁹ to 10¹⁴ vg/kg, 10¹⁰ to 10¹³ vg/kg, 10¹⁰ to 10^{12.5} vg/kg, 10¹⁰ to 10¹² vg/kg, 10¹⁰ to 10^{11.5} vg/kg, or 10¹⁰ to 10¹¹ vg/kg. In some embodiments, the therapeutically effective amount of AAV vector is about or no more than 10⁶ vg/kg, 10⁷ vg/kg, 10⁸ vg/kg, 10⁹ vg/kg, 10¹⁰ vg/kg, 10¹¹ vg/kg, 10^{11.5} vg/kg, 10¹² vg/kg, 10^{12.5} vg/kg, or 10¹³ vg/kg. The therapeutically effective amount of a viral vector can vary depending on many factors, such as the type of viral vector, the cells to be transfected, the condition to be treated, the subject receiving the treatment (e.g. the disease state, age, sex, and body weight), the ability of the viral vector to elicit a desired response in the individual, and so on. The therapeutic effective amount may be determined by starting with a low but safe dose and escalating to higher doses, while monitoring for therapeutic effects (e.g. a reduction in cancer cell growth) along with the presence of any deleterious side effects.

In certain embodiments, the therapeutically effective amount in the treatment methods provided herein is a sub-therapeutic amount. The term “sub-therapeutic amount” as used herein refers to an amount of the exogenous nucleic acid that is lower than the conventional amount that would be required to produce a therapeutic benefit in a conventional treatment method where the subject is not administered with an aspirin compound either prior to or concurrently with the delivery of the exogenous nucleic acid (“conventional treatment method”).

In certain embodiments, a sub-therapeutic amount of exogenous nucleic acid produces insufficient, or does not produce any, therapeutic effect in a conventional treatment method (where no aspirin compound is administered).

In some embodiments, the sub-therapeutic amount is no more than 90%, no more than 80%, no more than 70%, no more than 60%, no more than 50%, no more than 40%, no more than 30%, no more than 20%, no more than 10%, no more than 5%, no more than 2%, or no more than 1% of the conventional amount of the same exogenous nucleic acid that would otherwise be required without the administration of the aspirin compound.

In certain embodiments, the therapeutically effective amount in the treatment methods provided herein is comparable or identical to the conventional amount, but provides for a higher therapeutic effect than a conventional treatment method. In certain embodiments, the methods provided herein can achieve therapeutic effects that are at least 10% higher, 20% higher, 30% higher, 40% higher, 50% higher, 60% higher, 70% higher, 80% higher, 90% higher, 100% higher, 150% higher, 200% higher, 250% higher, 300% higher, 350% higher, 400% higher, 450% higher, or 500% higher than that can be achieved by the conventional treatment method.

In some embodiments, the aspirin compound can be administered to the subject prior to or concurrently with the administration of the exogenous nucleic acid.

In some embodiments, the aspirin compound is administered to the subject at least 1 day, 2 days, 3 days, 4 days, 5 days, 6 days or 7 days prior to the delivery of the exogenous nucleic acid to the subject. In some embodiments, the aspirin compound is administered to the subject for once or repetitively (e.g. twice, three times, four times and so on) prior to the delivery of the nucleic acid. In some embodiments, the aspirin compound is administered to the subject concurrently with the exogenous nucleic acid.

In some embodiments, the aspirin compound is administered at an amount sufficient to effectively increase expression of the exogenous nucleic acid in the cell or in the subject. In certain embodiments, the aspirin compound is administered at an effective amount to provide for at least 50%, 60%, 70%, 80%, 90%, 100%, 150%, 200%, 250%, 300% or more increase in expression of the exogenous nucleic acid in the subject.

In some embodiments, the aspirin compound is administered to the subject at an amount of no more than 30 mg/kg, no more than 50 mg/kg, no more than 60 mg/kg, no more than 70 mg/kg, no more than 80 mg/kg, no more than 90 mg/kg, no more than 100 mg/kg, no more than 110 mg/kg, no more than 120 mg/kg, no more than 120 mg/kg, no more than 130 mg/kg, no more than 140 mg/kg, no more than 150 mg/kg, no more than 160 mg/kg, no more than 170 mg/kg, no more than 180 mg/kg, no more than 190 mg/kg, or no more than 200 mg/kg. In some embodiments, the aspirin compound is administered to the subject at an amount from 30 mg/kg to 200 mg/kg, from 30 mg/kg to 150 mg/kg, from 30 mg/kg to 120 mg/kg, from 30 mg/kg to 100 mg/kg, from 30 mg/kg to 90 mg/kg, from 30 mg/kg to 80 mg/kg, from 30 mg/kg to 70 mg/kg, from 30 mg/kg to 60 mg/kg, from 30 mg/kg to 50 mg/kg, from 30 mg/kg to 40 mg/kg. In certain embodiments, the aspirin compound is administered to the subject at an amount from 30 mg/kg to 50 mg/kg, for example, at about 35 mg/kg, 40 mg/kg, 45 mg/kg, or 50 mg/kg, In certain embodiments, the aspirin compound is administered to the subject at an amount from 50 mg/kg to 100 mg/kg, for example, at about 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg.

In some embodiments, the aspirin compound is administered to the subject concurrently with the exogenous nucleic acid. In certain embodiments, the aspirin compound and the exogenous nucleic acid can be administrated sufficiently close in time (for example simultaneously, or within a short time period before or after each other), such that they are expected to take effect in the subject at substantially the same time. The aspirin compound and the exogenous nucleic acid can be considered as administered concurrently as long as the two agents enter into cells within a short time period before, after or simultaneously with each other. In some embodiments, the aspirin compound is administered concurrently with the exogenous nucleic acid in the form of one single composition. In some other embodiments, the aspirin compound is administered concurrently with the exogenous nucleic acid in different compositions.

In some embodiments, the aspirin compound and the exogenous nucleic acid are mixed before concurrent administration to the subject. In some embodiments, the aspirin compound and the exogenous nucleic acid are co-administered to the subject intravenously. In some embodiments, the aspirin compound and the exogenous nucleic acid is administered to the subject through different administration route. In some embodiments, the aspirin compound is administered orally, intravenously, intraperitoneally, intramuscularly, subcutaneously, intrathecally, intra-cerebroventricularly (ICV), or intrarectally to the subject.

In some embodiments, the aspirin compound and/or the exogenous nucleic acid are administered to treat a Central Nervous System (CNS) disorder. In some embodiments, the CNS disorder is treated by systemic administration of the aspirin compound and/or the exogenous nucleic acid. In certain embodiments, the exogenous nucleic acid suitable for systemic administration for treating CNS disorder comprises an AAV vector (e.g. AAV virus particle), for example, an AAV9 vector (e.g. AAV9 particle). In certain embodiments, the systemic administration includes intravenous, subcutaneous, or intramuscular administration. In some embodiments, the CNS disorder is treated by intra-cerebroventricular, or intrathecal administration of the aspirin compound and/or the exogenous nucleic acid. A CNS disorder can be any condition that is treatable by delivering a therapeutic nucleic acid to brain or spinal cord. Examples of CNS disorders include for example, Parkinson's disease, Alzheimer's disease, Mucopolysaccharidosis type II, Mucopolysaccharidosis type IIIA, Mucopolysaccharidosis type IIIB, Huntington disease, amyotrophic lateral sclerosis, Epilepsy, Batten Disease, Spinocerebellar Ataxia, spinal muscular atrophy, Canavan disease, and Friedreich's ataxia.

Without wishing to be bound by any theory, it is believed that the present invention is particularly advantageous in treating CNS disorders, and this is at least partially attributable to the increased expression of exogenous nucleic acid in the target site of the brain. It was surprisingly found by the inventors that, by systematic administration of AAV in combination with aspirin compound provided herein, a much lower dose of AAV can be used yet achieving the same or even higher efficacy than a higher dose. It is known in the art that AAV9, if administered via a systematic route, must achieve a threshold dose of 10¹⁴ vg/kg in order to enable transgene expression in the brain, however, such a dosage is both technically challenging and costly to manufacture, and this significantly limits the use of AAV9 in treating CNS disorders (see, for details, Perez B A et al., Brain Sci. 2020 Feb. 22; 10(2). pii: E119; Duque S et al., Mol Ther. 2009 July; 17(7):1187-96; Foust K D et al., Nat Biotechnol. 2009 January; 27(1):59-65.) As disclosed in the present application, AAV9 delivered via a systematic route at an amount lower than 10¹⁴ vg/kg (e.g. at 10¹³ vg/kg, 10^{12.5} vg/kg, 10¹² vg/kg, 10¹¹ vg/kg or even lower) in combination with an aspirin compound, can enable transgene expression in brain, and is equally effective or even more effective as compared to AAV9 administered at or above 10¹⁴ vg/kg but without an aspirin compound. This can reduce the dose of AAV required for treating CNS disorders, and thereby reducing the complexity of manufacture process, making it possible to use for example lower dose AAV preparations to treat brain disorders.

In another aspect, the present disclosure provides a method of reducing adverse effects or improving tolerance to an exogenous nucleic acid in a subject. As used herein, an “adverse effect” caused by the delivery of an exogenous nucleic acid may be any kind of adverse effect known in the art with respect to drug delivery, including but are not limited to, nausea, vomiting, dizziness, somnolence/sedation, allergy, pruritus, reduced gastrointestinal motility including constipation, difficulty in urination, peripheral vasodilation including leading to orthostatic hypotension, headache, dry mouth, sweating, asthenia, dependence, mood changes (e.g., dysphoria, euphoria), lightheadedness, or even respiratory depression, apnea, respiratory arrest, circulatory depression, hypotension or shock.

In some embodiments of the present disclosure, the exogenous nucleic acid is delivered to the subject at a sub-therapeutic amount. In certain embodiments, the sub-therapeutic amount is therapeutically effective in the treatment methods provided herein but causes significantly reduced adverse effects than a conventional amount.

In some embodiments, the adverse effect is dose-dependent to the exogenous nucleic acid delivered to the subject. It is believed that, by administering the exogenous nucleic acid at a sub-therapeutic level, the adverse effects associated with the exogenous nucleic acid can be reduced as a result of less exposure.

### Composition

In yet another aspect, the present disclosure provides a composition comprising an aspirin compound and an exogenous nucleic acid in combination, wherein the exogenous nucleic acid comprises double-stranded DNA, or can be converted to double-stranded DNA in a cell after delivery of the exogenous nucleic acid to the cell.

In some embodiments, the exogenous nucleic acid in the composition comprises an encoding sequence that encodes for a protein of interest or a portion thereof, or that encodes for a functional RNA or a portion thereof.

In some embodiments, the therapeutic protein is selected from the group consisting of: SMN, NAGLU, SGSH, IDS, FVIII, FIX, BTK, ABCD1, ACADVL, AR, HBB, SCN1A, CFTR, CSF2RA, IL2AG, PHA, STK11, PIGA, OTC, NAGS, DMPK, CNBP, ACADM, GNAS, FBN1, LIPA, SLC7A7, HADHA, GHR, IDV, ALPL, SLC25A15, HTT, HCS, NOTCH3, ALDOB, ATP7B, GAA, GCDH, SLC12A3, GBA, MEFV, GLA, CLCN1 NR0B1, ASS1, SLC25A13, SLC22A5, SCN5A, BTD, ACAT1, ARG1, CYP21A2, a chimeric antigen receptor (CAR), an antibody (e.g. monoclonal or bispecific or multi-specific), insulin, glucagon-like peptide-1, peptide hormones, growth factors, erythropoietin (EPO), cytokines, coagulation factors, antihemophilic factors, interferons, Fc-fusion proteins (such as CTLA-4 Fc-fusion, VEGFR Fc-fusion), and therapeutic enzymes (e.g. lysosomal hydrolase, and sulfatases).

In some embodiments, the immunogenic protein is selected from the group consisting of an immunogenic protein from orthomyxovirus (e.g. influenza virus), lentivirus (e.g. HIV, SIV), arenavirus (Lassa fever virus), poxvirus (e.g. vaccinia), flavivirus (e.g. yellow fever virus), filovirus (Ebola virus), bunyavirus (RVFV, CCHF, or SFS viruses), coronavirus (e.g. SARS, MERS, or COVID-19), poliovirus, herpes virus (CMV, EBV, HSV), mumps virus, measles virus, rubella virus, diphtheria toxin, pertussis, and hepatitis virus (e.g. HAV, HBV, or HCV).

In some embodiments, the nuclease comprises a Zinc-finger nucleases (ZFN), a transcription activator-like effector nucleases (TALEN), or a Cas family protein (such as Cas1, Cas1B, Cas2, Cas3, Cas4, Cas5, Cas6, Cas7, Cas8, Cas9 (also known as Csn1 and Csx12), Cas 10, Cas 11, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologues thereof, or modified versions thereof.

In some embodiments, the reporter proteins is selected from the group consisting of: a fluorescent protein (e.g., EGFP, GFP, RFP, BFP, YFP, or dsRED2), an enzyme that produces a detectable product, such as luciferase (e.g., from Gaussia, Renilla, or Pho firms), b-galactosidase, b-glucuronidase, alkaline phosphatase, chloramphenicol acetyltransferase gene, and proteins that can be directly detected.

In some embodiments, the therapeutic target protein (e.g. CTLA-4, HER2, Nectin-4, Sclerostin, P-Selectin, VEGF, RSVF, VEGFR2, CD79, IL23p19, vWF, IFN-γ, C5, PD-1, PD-L1, CGRP, CD3, CDiia, CD20, CD22, CD30, CD33, CD38, CD40, CD52, IgE, KLK, CCR4, FGF-23, IL-6R, IL-5, IL-23p19, IL-2R, IL-17R, IL-17, CD4, FIX/FX, IL-12, IL-23, IL-1β, IL-5R, IL-6R, IL-4/IL-13, PDGF-α, Dabigatran, SLAMF7, EGFR, PCSK9, GD2, CD3, CD19, α4β7 integrin, α4β1 integrin, PA, BLyS, RANK, TNF-α, EpCAM, GGTA1, Endostatin, Angiostatin).

In some embodiments, the functional RNA modulates a biological target selected from the group consisting of: multiple drug resistance (MDR) protein target, a tumor target (e.g. VEGF, Her2, EGFR, PD-L1 and so on), a pathogen target such as a viral surface antigen (e.g. hepatitis B surface antigen gene), a defective gene product (mutated dystrophins), or a therapeutic target (e.g. myostatin).

### Pharmaceutical Composition

In yet another aspect, the present disclosure provides a pharmaceutical composition comprising a sub-therapeutic amount of the exogenous nucleic acid provided herein, and a pharmaceutically acceptable carrier. In certain embodiments, the pharmaceutical composition further comprises an aspirin compound provided herein.

In yet another aspect, the present disclosure provides a pharmaceutical composition comprising the exogenous nucleic acid provided herein, an aspirin compound provided herein, and a pharmaceutically acceptable carrier.

The term “pharmaceutically acceptable carrier” as used herein refers to any and all pharmaceutical carriers, such as solvents, dispersion media, coatings, antibacterial and antifungal agents, isotonic and absorption delaying agents, and the like that can facilitate storage and administration of the nucleic acids, expression vectors of the present disclosure to a subject, and/or to the host cells. In certain embodiments, the host cells that have been administered with the pharmaceutical composition are suitable for administration to a subject. The pharmaceutically acceptable carriers can include any suitable components, such as without limitation, saline, liposomes, polymeric excipients, colloids, or carrier particles.

In certain embodiments, the pharmaceutically acceptable carriers are saline that can dissolve or disperse the nucleic acids, expression vectors, and/or host cells of the present disclosure. Illustrative examples of saline include, without limitation, buffer saline, normal saline, phosphate buffer, citrate buffer, acetate buffer, bicarbonate buffer, sucrose solution, salts solution and polysorbate solution.

In certain embodiments, the pharmaceutically acceptable carriers are liposomes. Liposomes are uni-lamellar or multi-lamellar vesicles, having a membrane formed of lipophilic material and an interior aqueous portion. The nucleic acids, expression vectors, and/or host cells of the present disclosure can be encapsulated within the aqueous portion of the liposomes. Illustrative examples of liposomes include, without limitation, liposomes based on 3[N—(N′,N′-dimethylaminoethane) carbamoyl] cholesterol (DC-Chlo), liposomes based on N-(2,3-dioleoyloxy)propyl-N,N,N-trimethylammonium chloride (DOTMA), and liposomes based on 1,2-dioleoyloxy-3-trimethylammonium propane (DOTAP). Methods of preparing liposomes and encapsulating the expression vectors in liposomes are well known in the art (see for example, D. D. Lasic et al, Liposomes in gene delivery, published by CRC Press, 1997).

In certain embodiments, the pharmaceutically acceptable carriers are polymeric excipients, such as without limitation, microspheres, microcapsules, polymeric micelles and dendrimers. The nucleic acids, expression vectors, and/or host cells of the present disclosure may be encapsulated, adhered to, or coated on the polymer-based components by methods known in the art (see for example, W Heiser, Nonviral gene transfer techniques, published by Humana Press, 2004; U.S. Pat. No. 6,025,337; Advanced Drug Delivery Reviews, 57(15): 2177-2202 (2005)).

In certain embodiments, the pharmaceutically acceptable carriers are colloids or carrier particles such as gold colloids, gold nanoparticles, silica nanoparticles, and multi-segment nanorods. The nucleic acids, expression vectors or cells may be coated on, adhered to, or associated with the carriers in any suitable manner as known in the art (see for example, M. Sullivan et al., Gene Therapy, 10: 1882-1890 (2003), C. McIntosh et al., J. Am. Chem. Soc., 123 (31): 7626-7629 (2001), D. Luo et al., Nature Biotechnology, 18: 893-895 (2000), and A. Salem et al., Nature Materials, 2: 668-671 (2003)).

In certain embodiments, the pharmaceutical composition may further comprise additives, such as without limitation, stabilizers, preservatives, and transfection facilitating agents which assist in the cellular uptake of the medicines. Suitable stabilizers may include, without limitation, monosodium glutamate, glycine, EDTA and albumin (e.g. human serum albumin). Suitable preservatives may include, without limitation, 2-phenoxyethanol, sodium benzoate, potassium sorbate, methyl hydroxybenzoate, phenols, thimerosal, and antibiotics. Suitable transfection facilitating agents may include, without limitation, calcium ions.

The pharmaceutical composition may be suitable for administration via any suitable routes known in the art, including without limitation, parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, or intrathecal administration routes.

The pharmaceutical composition can be administered to a subject in the form of formulations or preparations suitable for each administration route. Formulations suitable for administration of the pharmaceutical composition may include, without limitation, solutions, dispersions, emulsions, powders, suspensions, aerosols, sprays, nose drops, liposome based formulations, patches, implants and suppositories.

The formulations may conveniently be presented in unit dosage form and may be prepared by any methods well known in the art of pharmacy. Methods of preparing these formulations or compositions include the step of providing the exogenous nucleic acid of the present disclosure to one or more pharmaceutically acceptable carriers and, optionally, one or more adjuvants. Methods for making such formulations can be found in, for example, Remington's Pharmaceutical Sciences (Remington: The Science and Practice of Pharmacy, 19th ed., A. R. Gennaro (ed), Mack Publishing Co., N.J., 1995; R. Stribling et al., Proc. Natl. Acad. Sci. USA, 89:11277-11281 (1992); T. W. Kim et al., The Journal of Gene Medicine, 7(6): 749-758(2005); S. F. Jia et al., Clinical Cancer Research, 9:3462 (2003); A. Shahiwala et al., Recent patents on drug delivery and formulation, 1:1-9 (2007); A. Barnes et al., Current Opinion in Molecular Therapeutics 2000 2:87-93(2000), which references are incorporated herein by reference in their entirety).

In certain embodiments, the pharmaceutical composition containing the exogenous nucleic acids (e.g. AAV vectors or AAV virus particles) of the present disclosure is suitable for administration to a subject in need of treatment through local delivery into the target tissue or organ at the diseased site. In certain embodiments, the pharmaceutical composition is suitable for directly injection to a diseased site palpable through the skin using a syringe. In certain embodiments, the pharmaceutical composition is suitable for injection using an implantable dosing device connected to a catheter line or other medical access device, and may be used in conjunction with an imaging system guiding to the diseased site. In certain embodiments, the pharmaceutical composition is suitable for direct injection in an effective dose to a diseased site visible in an exposed surgical field. In certain embodiments, the pharmaceutical composition (e.g. vector-coated gold particles) is suitable for bombardment to the diseased site using a gene gun which shots the particles directly into the diseased site (see, for example, R. Muangmoonchai et al., Molecular Biology, 20(2):145-151(2002)). In certain embodiments, the pharmaceutical compositions are suitable for administration to a subject through intravenous injection. In certain embodiments, the pharmaceutical compositions are suitable for administration orally or transmucosally to a subject. For references of methods of introducing therapeutic nucleic acids into cells or animals, see, for example, Yang, N-S., Crit. Rev. Biotechnol. 12:335-356 (1992); Anderson, W F., Science 256:808-813 (1992); Miller, A. S., Nature 357:455-460 (1992); Crystal, R. G., Amer. J. Med. 92 (suppl 6A):44S-52S (1992); Zwiebel, J. A. et al., Ann. N.Y Acad. Sci. 618:394-404 (1991); McLachlin, J. R. et al., Prog. Nucl. Acid Res. Molec. Biol. 38:91-135 (1990); Kohn, D. B. et al., Cancer Invest. 7:179-192 (1989). These references are incorporated herein by reference in their entirety.

In some embodiments, the exogenous nucleic acid in the pharmaceutical composition comprises an encoding sequence that encodes for a protein of interest or a portion thereof, or that encodes for a functional RNA or a portion thereof. In some embodiments, the exogenous nucleic acid in the pharmaceutical composition comprises a nucleic acid of interest encoding for a therapeutic protein. In some embodiments, the exogenous nucleic acid in the pharmaceutical composition comprises a nucleic acid of interest encoding for a therapeutic protein suitable for treating a CNS disorder or a brain disease. In some embodiments, the therapeutic protein or therapeutic target for a functional RNA for treating a CNS disorder or a brain disease include, for example without limitation, Tau, MeCP2, NGF, APOE, GDNF, SUMF, SGSH, AADC, CD, p53, ARSA arylsulfatase A, ABCD1, SMN1, NAGLU, SOD1, C9ORF72, TARDBP, FUS, HTT, LRRK2, PARIS, PARKIN, GAD, and α-synuclein. These genes are known in the art, and have been described in. for example, Maguire C A et al., Neurotherapeutics. 2014 October; 11(4):817-39; Bowers W J et al., Hum Mol Genet. 2011 Apr. 15; 20(R1):R28-41).

In some embodiments, the exogenous nucleic acid comprises an AAV vector (e.g. AAV virus particle). In some embodiments, the AAV virus particle in the pharmaceutical composition is suitable for providing a dose at an amount no more than 10¹⁴ vg/kg (e.g. no more than 10¹³ vg/kg, 10^{12.5} vg/kg, 10¹² vg/kg, 10^{12.5} vg/kg, 10¹² vg/kg, 10^{11.5} vg/kg, 10¹¹ vg/kg, 10^{10.5} vg/kg, 10¹⁰ vg/kg, or 10⁹ vg/kg). In some embodiments, the AAV virus particle in the pharmaceutical composition is suitable for providing a sub-therapeutic dose.

In some embodiments, the pharmaceutical composition is in a unit dose, and contains no more than 10¹⁰ vg, 10^{10.5} vg, 10¹¹ vg, 10^{11.5} vg, 10¹² vg, 10^{12.5} vg, 10¹³ vg, 10^{13.5} vg, 10¹⁴ vg, 10^{14.5} vg, 10¹⁵ vg, 10^{15.5} vg, or 10¹⁶ vg of AAV virus particle. “Unit dose” as used herein is the dose that is sufficient to provide for one treatment. In some embodiments, the unit dose is for human use, for example, for human adult (e.g. average body weight of 60 kg), human adolescent, human child, or human infant. In some embodiments, the pharmaceutical composition is in a formulation suitable for systemic administration, such as, for intravenous injection, intravenous infusion, and intramuscular injection.

In some embodiments, the aspirin compound is packaged together with AAV particle, for example in the form of a mixture or in one composition. In some embodiments, the aspirin compound is packaged independently, separated from the AAV virus particle, for example, the aspirin compound can be provided in any commercially available form in a separate container.

In some embodiments, the pharmaceutical composition of the present disclosure further comprises an instruction for use that indicates the aspirin compound is to be administered prior to or concurrently with administration of the pharmaceutical composition. In some embodiments, the instructions for use indicates that for treating brain diseases, the AAV virus is administered through systemic administration, preferably at a dose lower than 10¹⁴ vg/kg. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

### Kit

In a further aspect, the present disclosure provides a kit comprising: a) a first composition comprising the aspirin compound; and b) a second composition comprising the exogenous nucleic acid provided herein. In some embodiments, a kit further comprises instructions for using the components of the kit to practice the methods of the present disclosure.

In certain embodiments, the first composition and the second composition are in separate containers. They can be combined before use, or can be used separately, for example at different timing or via different administration routes.

In a further aspect, the present disclosure provides a kit comprising a composition comprising the aspirin compound and the exogenous nucleic acid provided herein. In some embodiments, a kit further comprises instructions for using the components of the kit to practice the methods of the present disclosure.

In some embodiments, the kit further comprises an instruction for use that indicates that the second composition is to be administered prior to or concurrently with the second composition. In some embodiments, the first composition and the second composition can be readily mixed to provide a combined composition before use. In some embodiments, the second composition comprises the exogenous nucleic acid at a sub-therapeutic amount.

In yet a further aspect, the present disclosure provides a composition comprising: an aspirin compound and a sub-therapeutic amount of the exogenous nucleic acid provided herein.

The following examples are presented to illustrate the present invention and to assist one of ordinary skill in making and using the same. The examples are not intended in any way to otherwise limit the scope of the invention.

### EXAMPLES

### Example 1

Unless otherwise indicated, the practice of certain examples described herein may employ conventional techniques of molecular biology, cell culture, recombinant nucleic acid (e.g., DNA) technology, immunology, and/or nucleic acid and polypeptide synthesis, detection, manipulation, and quantification, etc., that are within the ordinary skill of the art. See, e.g., Ausubel, F., et al., (eds.), Current Protocols in Molecular Biology, Current Protocols in Immunology, Current Protocols in Protein Science, and Current Protocols in Cell Biology, all John Wiley & Sons, N.Y, e.g., edition current as of January 2010 or later; Sambrook, Russell, and Sambrook, Molecular Cloning: A Laboratory Manual, 3^{rd} ed., Cold Spring Harbor Laboratory Press, Cold Spring Harbor, 2001 or 4^{th} ed, 2012.

Materials

The vector plasmid AAV-Lxp3.3-Gluc is prepared based on methods disclosed in U.S. patent publication US20160229904. In brief, synthetic promoter Lxp3.3 was synthesized based on the nucleotide sequence as disclosed in US20160229904 (also provided herein as SEQ ID NO: 1), and the LXP3.3 promoter was linked at its 3′ end to the reporter gene Gaussia luciferase (Gluc) encoding gene (sequence of which can be found at GenBank accession number: LC006266.1, see also, SEQ ID NO: 2), to obtain the Lxp3.3-Gluc expression cassette. The Lxp3.3-Gluc expression cassette was inserted between the AAV2 ITRs in an AAV expression plasmid, and was co-transfected into HEK 293 cells with a second plasmid expressing Rep gene, and a third plasmid encoding Cap gene of AAV8, AAV9, or AAV843, to be packaged into AAV8-Lxp3.3-Gluc, AAV9-Lxp3.3-Gluc, or AAV843-Lxp3.3-Gluc viruses, respectively. The Cap gene of AAV8 was shown in SEQ ID NO: 4 (obtained from GenBank Accession number: AF513852), and the encoded Cap protein sequence is shown in SEQ ID NO: 8 (see, also, GenBank Accession Number AAN03857.1). Cap gene of AAV9 from GenBank Accession number: AY530579 (see, also SEQ ID NO: 5), and the encoded Cap protein sequence is shown in SEQ ID NO: 9. Cap gene of AAV843 from the synthetic capsid gene sequence of AAVXL32 as disclosed in WO2019241324A1 (see also, SEQ ID NO: 6), and the encoded Cap protein sequence is shown in SEQ ID NO: 10. Virus titers were quantified by real-time PCR, and dot-blot, using commercially available kits and based on manufacturer's instructions. Among them AAV8-Lxp3.3-Gluc, AAV9-Lxp3.3-Gluc were produced, showing titers of 2.67×10¹³ (vector genome/mL) vg/mL and 2.99×10¹³ vg/mL, respectively. AAV843-Lxp3.3-Gluc was produced in-house with a titer of 4×10¹² vg/mL.

Acetylsalicylic acid with CAS number: 50-78-2 (aspirin) was purchased from Shanghai Aladdin Biochemical Technology Co., Ltd.

### Example 2

1.1 Aspirin Treatment and Viral Vector Delivery

Male C57BL/6J mice (purchased from Shanghai Jiesijie Experimental Animals Co., Ltd.) (n=4) with an average weight of 20 g were divided into six groups. Aspirin was used for intraperitoneal injection for 7 consecutive days (Day −7 to Day −1), at doses of 1 mg/kg, 15 mg/kg, 30 mg/kg, 50 mg/kg, 100 mg/kg, respectively, to five groups of mice. The sixth group was injected with 5% castor oil (in PBS buffer (catalogue number: 02-024-1ACS, BI)) as a control. On Day 0, AAV9-Lxp3.3-Gluc virus was injected intravenously (5×10¹² vg/kg) to each mouse in every group, and expression of luciferase was detected from Day 1 onwards.

1.2 Sample Collection

Blood samples of mice were collected by retro-orbital bleeding. Half of the volume of blood samples was added with sodium citrate at a ratio of 1:9 for anticoagulation, mixed, centrifuged within 30 minutes (6000 rpm, 5 mins), and the supernatant was collected as the mouse plasma, for detection of the expression of Gluc. The remaining blood sample was left at room temperature for 2 hours to separate the serum, centrifuged at 6000 rpm for 15 min, and the supernatant was collected as the mouse serum sample. The contents of IFN-α and IFN-β were determined by using Mouse IFN-β ELISA kit (720131-2, Shanghai Mlbio Co., Ltd.), LEGEND MAX™ Mouse IFN-β ELISA Kit (439407, BioLegend), and Mouse IFN-α ELISA kit (706291-2, Shanghai Mlbio Co., Ltd.).

1.3 Detection of Gluc

Coelenterazine h (40906ES02, Shanghai Yeasen Biotech Co., Ltd.) buffer was prepared first: to 500 mL of ultra-pure water was added 29.72 g of sodium ascorbate, and then 50 mL of Tris-HCl (1 mol/L, pH 7.4). The stock solution of coelenterazine h was at a concentration of 200 NM. The working solution of coelenterazine h was prepared at a ratio of 1:1, and used as freshly prepared. 10 NL of the above working solution is added to a 10 L plasma sample, and the relative light unit (RLU) was detected with a Synergy H1 multifunctional microplate reader.

1.4 Results

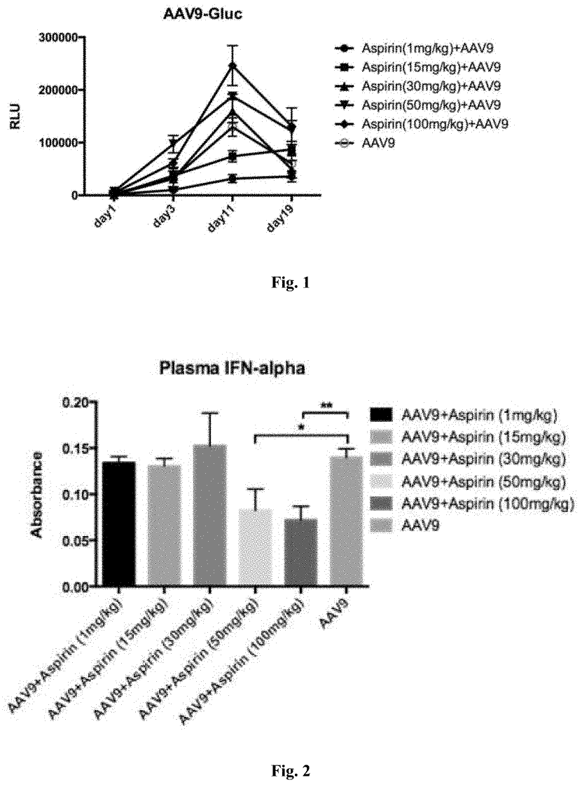

The results showed that the expression level of luciferase increased gradually over time in all groups, except that in some groups the expression level declined to some extent from Day 11. The expression level of luciferase in aspirin pre-treated groups showed dose dependent increase with aspirin concentration, as revealed by comparison across groups administered with different concentrations of aspirin, among which the highest expression level of luciferase was achieved at 100 mg/kg of aspirin (FIG. 1). The level of IFN-α on Day 3 was found to be significantly lower in the groups with aspirin treatment at 50 mg/kg and 100 mg/kg than in the control group (FIG. 2).

### Example 3

The injection dose of 50 mg/kg aspirin was used in further study of AAV-mediated expression of exogenous genes by three different AAV serotypes. The study design was similar to that of Example 2. Briefly, for each AAV serotype (i.e., AAV8-Lxp3.3-Gluc, AAV9-Lxp3.3-Gluc, or AAV843-Lxp3.3-Gluc), male C57BL/6J mice (purchased from Shanghai Jiesijie Experimental Animals Co., Ltd.) (n=4) with an average weight of 20 g were divided into two groups. One group was injected with aspirin at 50 mg/kg for 7 consecutive days, and the other group was injected with 5% castor oil (in PBS buffer (catalogue number: 02-024-1ACS, BI)) as a control. On Day 8, 5×10¹² vg/kg of AAV8-Lxp3.3-Gluc, AAV9-Lxp3.3-Gluc, or AAV843-Lxp3.3-Gluc virus (diluted in 150-200 μl PBS) was delivered to each mouse by tail vein injection. Blood sample were collected and expression level of luciferase was detected following the same methods as described in Example 2.

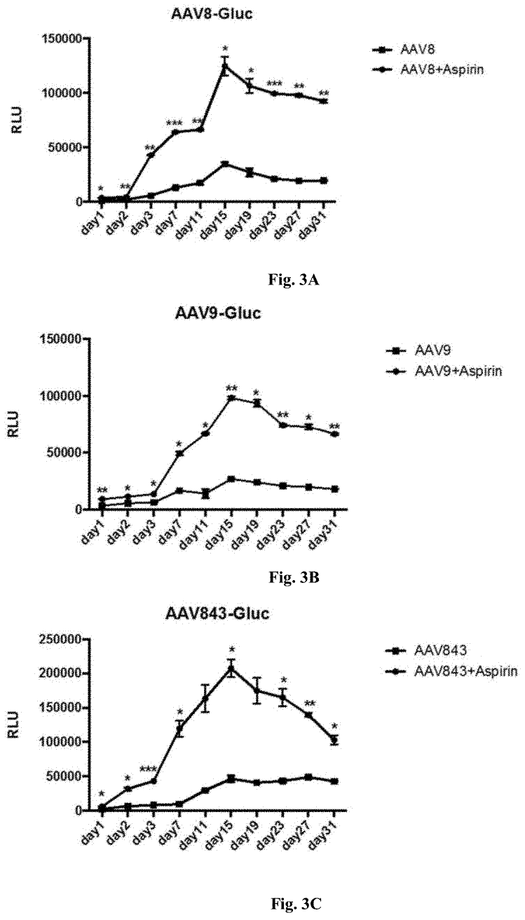

The results showed that aspirin could remarkably promote the transduction of all the tested virus serotypes, namely AAV8-Lxp3.3-Gluc, AAV9-Lxp3.3-Gluc, and AAV843-Lxp3.3-Gluc virus in vivo. Expression of the luciferase gene contained in the three AAV serotypes showed significant differences right from Day 1 after AAV injection, as compared to the control group. The expression of luciferase increased rapidly over time, peaked on Day 15 after AAV injection, and then decreased, across the entire period of which aspirin treatment groups showed significant differences from their corresponding control groups (FIGS. 3A, 3B, 3C). The expression of luciferase in the control groups also peaked on Day 15.

Studies have been conducted for co-administration of AAV and aspirin either in one formulation or in separate formulations but at the same time. The preliminary results showed that co-administration of AAV and aspirin could also increase the expression level of the transgene.

In conclusion, the results showed that, proper treatment of mice with aspirin before or concurrently with AAV injection can remarkably promote AAV transduction, increase the expression level of transgenes, and the increase in transgene expression has no serotype dependency.

### Example 4

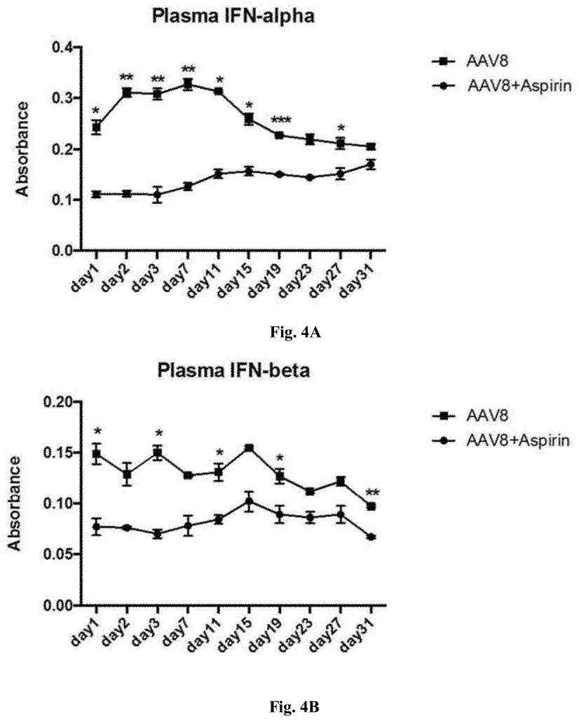

In the study described in Example 3, the level of IFN-α and IFN-β were determined by using Mouse IFN-β ELISA kit (720131-2, Shanghai Mlbio Co., Ltd.), LEGEND MAX™ Mouse IFN-β ELISA Kit (439407, BioLegend), and Mouse IFN-α ELISA kit (706291-2, Shanghai Mlbio Co., Ltd.) according to the user's manual. Analysis of IFN-α and IFN-β levels showed that aspirin remarkably inhibited the expression of type I interferon in mice after injection of AAV8, AAV9, and AAV843. The IFN-α level in the control group for AAV8 injection increased significantly from Day 2, and gradually decreased from Day 15, while the IFN-α maintained at a relatively low level in mice treated with aspirin, though showing some elevation from Day 3 to Day 11 and from Day 23 to Day 31 after injection of AAV8 (FIG. 4A). Except for Day 31, the IFN-α level in the control group was significantly higher than that in the aspirin treatment group. The IFN-3 level in the control group for AAV8 injection was significantly higher than that in the aspirin treatment group. The IFN-β level in the control group gradually decreased over time, but always significantly higher than that in the aspirin treatment group (FIG. 4B).

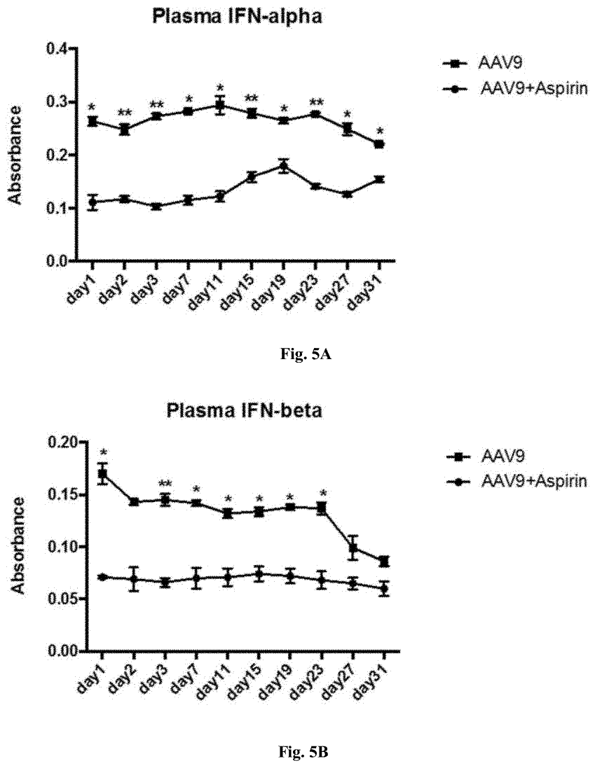

After injection of AAV9, the IFN-α level in the aspirin treatment group increased from Day 11 to Day 18, but were significantly lower than that in the control group (FIG. 5A). In the control group, IFN-α started to decrease gradually from Day 23 (FIG. 5A). Similar to the trend of the IFN-α level in the control group, the IFN-β level in the control group also decreased significantly after Day 23, while in the aspirin treatment group, IFN-β maintained at a low level from Day 1 to Day 31 and showed statistically significant differences from that of the control group from Day 1 to Day 23 (FIG. 5B).

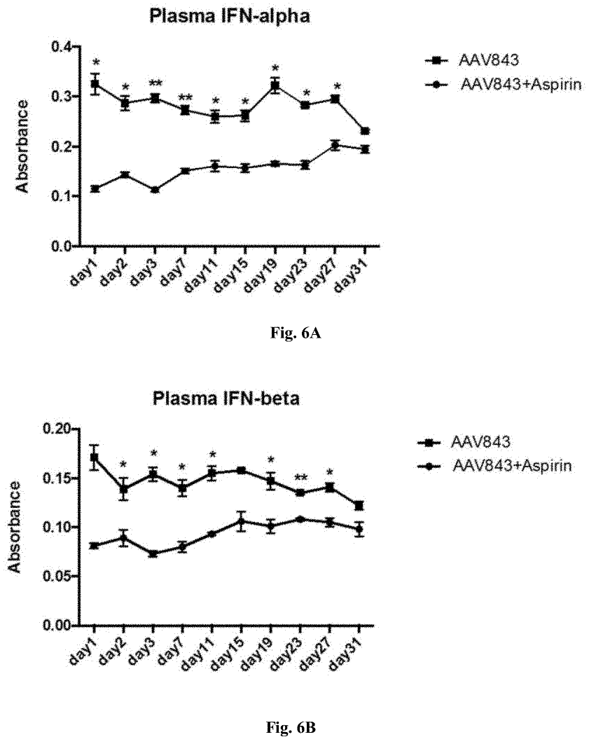

Measurement of IFN-α and IFN-β levels after injection of AAV843 showed that both the two type I interferons gradually increased over time in the aspirin treatment group, but their levels were significantly lower than those in the control group (FIG. 6A and FIG. 6B). The level of type I interferons in the control group showed a gradual decline. On Day 31 after the injection of virus, there was no significant difference in the levels of type I interferons between the aspirin treatment and the control groups (FIG. 6A and FIG. 6B).

In conclusion, the results showed that, aspirin can remarkably inhibit the activation of type I interferons after transduction AAVs of different serotypes, indicating that such effects have no serotype dependency.

### Example 5

To confirm the transgene expression in brain, male C57BL/6J mice (purchased from Shanghai Jiesijie Experimental Animals Co., Ltd.) (n=6-8 mice per group) with an average weight of 20 g are divided into five groups. Four groups are injected with aspirin at 50 mg/kg for 7 consecutive days first, and on Day 8, 10¹⁰ vg/kg, 10¹¹ vg/kg, 10¹² vg/kg, and 10¹³ vg/kg of AAV virus are respectively delivered to mice in the respective group by tail vein injection. The fifth group is a control group, which is injected with 5% castor oil (in PBS buffer (catalogue number: 02-024-1ACS, BI)) for 7 consecutive days, and on Day 8, 10¹⁴ vg/kg of AAV only is delivered to each mouse in the group. The animals are anesthetized 15-25 days post-injection and transcardially perfused, the brain of each mouse is fixed and then sectioned. Transgene expression is analyzed in different regions of brain. It is expected that intravenous administration of aspirin and AAV effectively leads to transgene expression in brain, with significantly increased CNS therapeutic effects. Interestingly, the dose of AAV used in the preliminary study is well below the dose (10¹⁴ vg/kg) that is believed to be capable of being expressed in brain. A pilot study showed that the transgene expression in brain was significantly increased, despite of the relatively low dose of AAV and low dose of aspirin.

Further studies are designed and in progress to further validate the CNS effects.

### Example 6

Expression of exogenous gene was detected in mice either pre-treated with Aspirin, or co-administered with Aspirin. AAV9-CB-Gluc was prepared according to methods provided in Example 1, except that nucleic acid sequence for CB promoter (see SEQ ID NO: 3) was used. In brief, Aspirin was pre-administrated to a group of mice at 50 mg/kg by intraperitoneal injection for 7 days, followed by administration of AAV9-CB-Gluc (5×10¹³ vg/kg) at day 8. In comparison, a group of mice without pre-administration of Aspirin was administrated with Aspirin at 50 mg/kg simultaneously in combination with AAV9-CB-Gluc. 15 days after the AAV9-CB-Gluc administration, the mice were sacrificed, and brain and liver were collected for determination of expression Gluc.

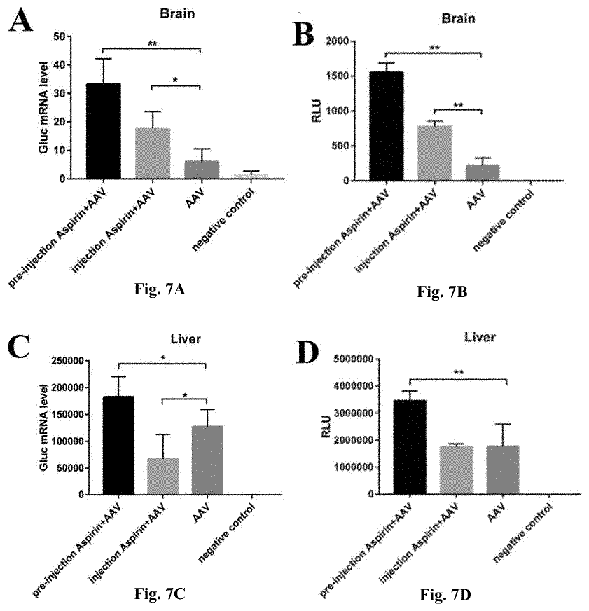

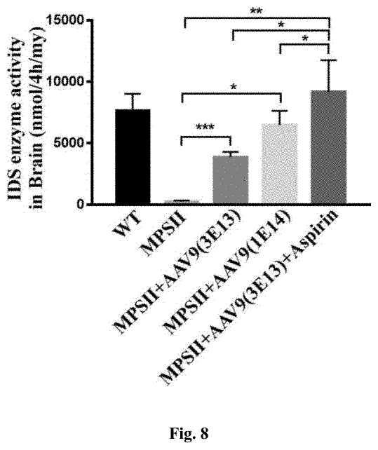

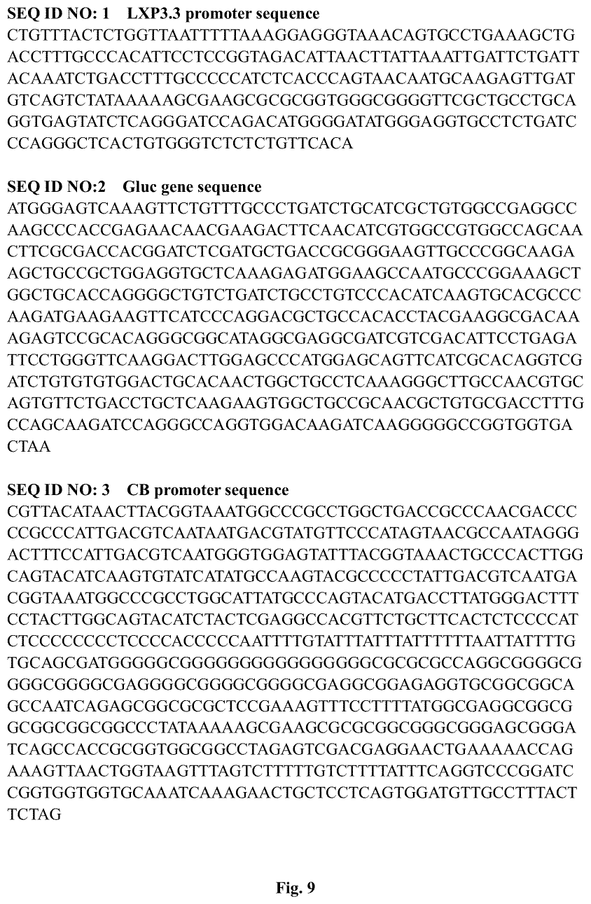

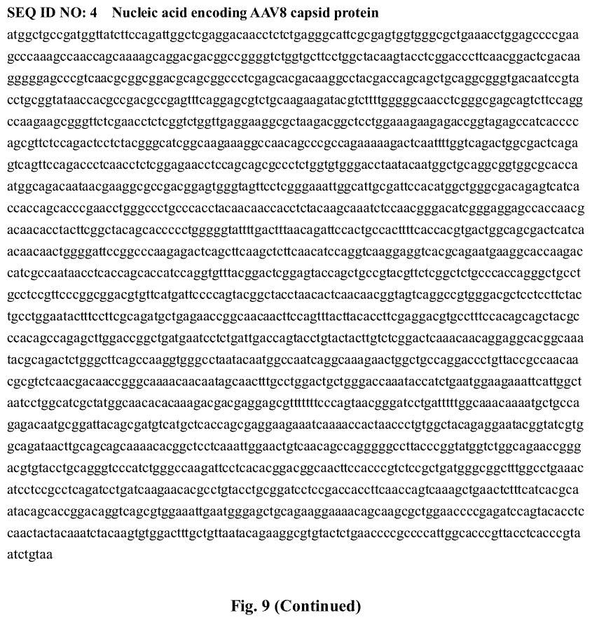

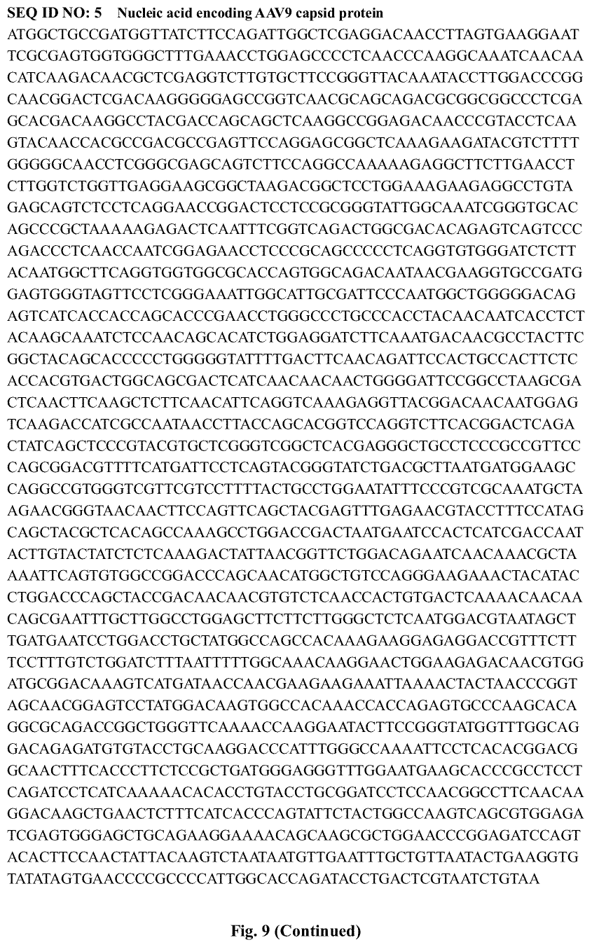

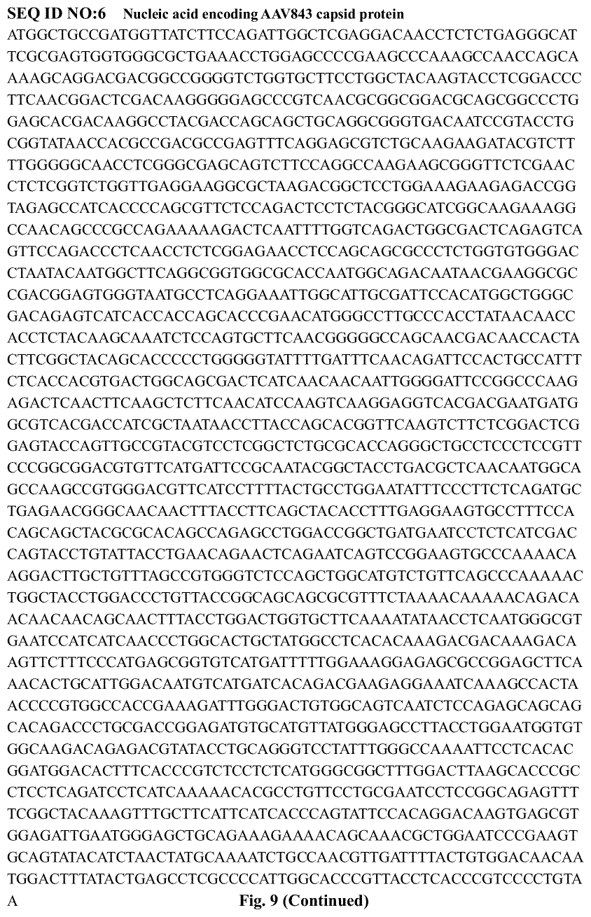

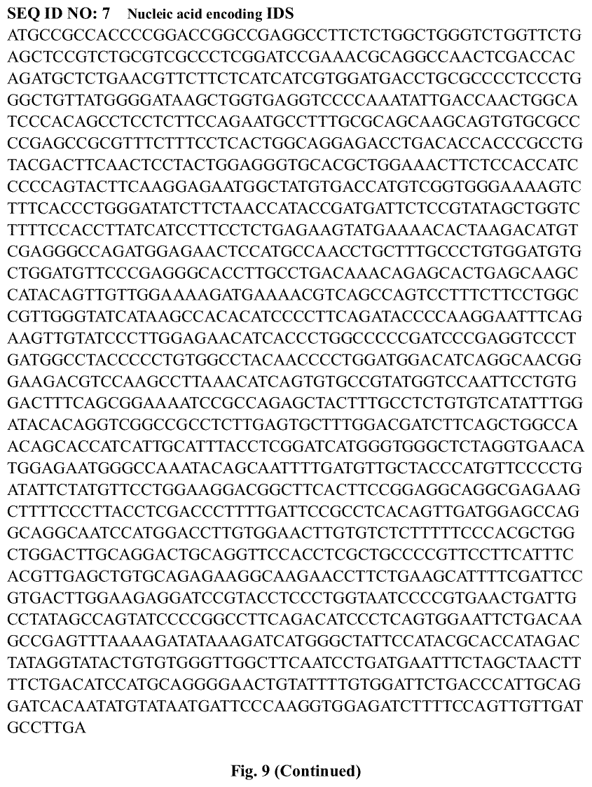

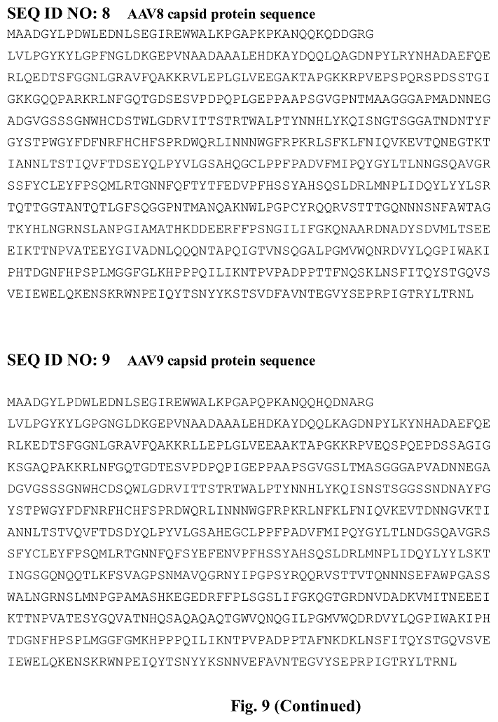

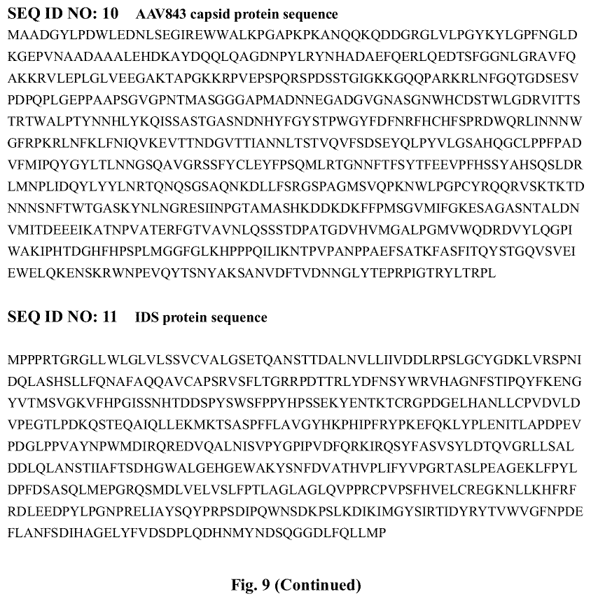

In brain, the mRNA level (FIG. 7A, p<0.01) and enzymatic activity level (FIG. 7B, p<0.01) of Gluc in mice receiving pre-injection of aspirin were up-regulated 5.5 fold and 7.13 fold, respectively, in comparison with the non-treatment group (i.e. mice administered with AAV9-CB-Gluc without any Aspirin treatment). In addition, in the simultaneous treatment group, the mRNA level (FIG. 7A, p<0.01) and enzymatic activity level (FIG. 7B, p<0.01) of Gluc level were also up-regulated 2.95 fold (p<0.05) and 3.55 fold (p<0.01) relative to the non-treatment group.

In liver, the level of mRNA and enzymatic activity in mice receiving pre-injection of aspirin were up-regulated 1.43 fold (FIG. 7C, p<0.05) and 1.95 (FIG. 7D, p<0.01) fold, respectively, in comparison with the non-treatment group. However, the simultaneous treatment group did not increase the level of mRNA or level of enzymatic activity in liver (FIGS. 7C, 7D, p<0.05) relative to the non-treatment group. The mRNA level was even lower than that of the non-treatment group (FIG. 7C), while the level of enzymatic activity was not significantly different (FIG. 7D).

In summary, Aspirin has been shown to significantly improve the transduction of AAV9 and increase expression of an exogenous gene in brain. Increase in expression of an exogenous gene in liver was also observed with aspirin pre-treatment group.

### Example 7

The effects of aspirin on improving the expression of therapeutic transgene in brain were determined. AAV9-CB-IDS vector were produced using methods described in Example 1, except that iduronate 2-sulfatase (IDS) gene expression cassette was inserted between the AAV ITRs. The gene encoding IDS was provided in SEQ ID NO: 7 and the protein sequence of IDS was provided in SEQ ID NO: 11. To determine therapeutic effects of the transgene, we used B6N.Cg-Ids^{tm1Muen}/J mice (n=5, obtained from JAX mice, cat No.: 024744) that was a Mucopolysaccharidosis II (MPSII) model having inactive Iduronate-2-sulfatase (IDS) due to mutation. A group of wild-type control mice was used as control. The AAV9-CB-IDS vector for treating MPSII was administrated as described in Example 1, at a dose of 3×10¹³ vg/kg (i.e. 3E13 group) or 1×10¹⁴ vg/kg (i.e. 1E14 group) to mice pre-treated with aspirin injection. One month after the AAV9-CB-IDS injection, the IDS enzyme activity was detected in brain (FIG. 8). The results showed that, in the 3E13 group, Aspirin pre-treatment significantly increased the IDS enzyme activity in the MPSII mice to a level even higher than that of the wild type mice group, and such level was significantly higher than the group receiving no aspirin but only AAV9-CB-IDS at the same dose (3×10¹³ vg/kg) (p<0.05) or even at a higher dose (10¹⁴ vg/kg). These results confirmed that aspirin could significantly decrease the dose of AAV that would otherwise be required for treating a disease without aspirin treatment.

What we claimed is:

1. A method of priming a cell for delivery of an exogenous nucleic acid, the method comprising:
administering an aspirin compound to the cell prior to or concurrently with the delivery of the exogenous nucleic acid to the cell,
wherein the exogenous nucleic acid comprises double-stranded DNA, or can be converted to double-stranded DNA in the cell after delivery.

2. A method of expressing an exogenous nucleic acid in a cell, the method comprising:
delivering to the cell the exogenous nucleic acid in a condition suitable for expression,
wherein the cell has been or is concurrently being administered with an aspirin compound; and
wherein the exogenous nucleic acid comprises double-stranded DNA, or can be converted to double-stranded DNA in the cell after delivery.

3. A method of expressing an exogenous nucleic acid in a cell, the method comprising:
a) administering an aspirin compound to the cell; and
b) delivering to the cell the exogenous nucleic acid in a condition suitable for expression,
wherein the step a) is prior to or concurrently with the step b),
wherein the exogenous nucleic acid comprises double-stranded DNA, or can be converted to double-stranded DNA in the cell after delivery.

4. A method of increasing expression level of an exogenous nucleic acid in a cell, the method comprising:
administering an aspirin compound to the cell prior to or concurrently with the delivery to the cell of the exogenous nucleic acid in a condition suitable for expression,
wherein the exogenous nucleic acid comprises double-stranded DNA, or can be converted to double-stranded DNA in the cell after delivery, and
whereby the expression level of the exogenous nucleic acid is increased as compared to a control expression level obtained in a control cell without the administration of the aspirin compound.

5. A method of increasing expression level of an exogenous nucleic acid in a cell, the method comprising:
delivering to the cell the exogenous nucleic acid in a condition suitable for expression,
wherein the cell has been or is concurrently being administered with an aspirin compound,
wherein the exogenous nucleic acid comprises a double-stranded DNA, or can be converted to double-stranded DNA in the cell after delivery, and
whereby the expression level of the exogenous nucleic acid is increased as compared to a control expression level obtained in a control cell without the administration of the aspirin compound.

6. A method of increasing expression level of an exogenous nucleic acid in a cell, the method comprising:
a) administering an aspirin compound to the cell; and
b) delivering to the cell the exogenous nucleic acid in a condition suitable for expression,
wherein the step a) is prior to or concurrently with the step b),
wherein the exogenous nucleic acid comprises a double-stranded DNA, or can be converted to double-stranded DNA in the cell after delivery, and
wherein the expression level of the exogenous nucleic acid is increased as compared to a control expression level obtained in a control cell without the step a).

7. A method of prolonging expression duration of an exogenous nucleic acid in a cell, the method comprising:
administering to the cell an aspirin compound prior to or concurrently with the delivery of the exogenous nucleic acid to the cell,
wherein the exogenous nucleic acid comprises double-stranded DNA, or can be converted to double-stranded DNA in the cell after delivery, and
wherein the expression duration of the exogenous nucleic acid in the cell is increased as compared to a control expression duration obtained in a control cell without the administration of the aspirin compound.

8. A method of prolonging expression duration of an exogenous nucleic acid in a cell, the method comprising:
delivering to the cell the exogenous nucleic acid in a condition suitable for expression,
wherein the cell has been or is concurrently being administered with an aspirin compound,
wherein the exogenous nucleic acid comprises double-stranded DNA, or can be converted to double-stranded DNA in the cell after delivery, and
wherein the expression duration of the exogenous nucleic acid in the cell is increased as compared to a control expression duration obtained in a control cell without the administration of the aspirin compound.

9. A method of prolonging expression duration of an exogenous nucleic acid in a cell, the method comprising:
a) administering an aspirin compound to the cell; and
b) delivering to the cell the exogenous nucleic acid in a condition suitable for expression,
wherein the step a) is prior to or concurrently with the step b),
wherein the exogenous nucleic acid comprises double-stranded DNA, or can be converted to double-stranded DNA in the cell after delivery, and
whereby the expression duration of the exogenous nucleic acid is prolonged as compared to a control expression duration obtained in a control cell without being administered with an aspirin compound.

10. The method of any one of claims 1-9, wherein the cell in in vitro, ex vivo, or in vivo.

11. The method of any one of claims 1-10, wherein the aspirin compound is administered to the cell at least 1 hour, 2 hours, 3 hours, 4 hours, 5 hours, 6 hours, 7 hours, 8 hours, 9 hours, 10 hours, 12 hours, 18 hours, 24 hours, 1.5 days, 2 days, 3 days, 4 days, 5 days, 6 days or 7 days prior to the delivery of the nucleic acid.

12. The method of any one of claims 1-11, wherein the aspirin compound is administered to the cell for once or repetitively (e.g. twice, three times, four times and so on) prior to the delivery of the nucleic acid.

13. The method of any one of claims 1-12, wherein the aspirin compound is administered at an amount sufficient to provide for at least 50%, 60%, 70%, 80%, 90%, 100%, 150%, 200%, 250%, 300% or more increase in expression of the exogenous nucleic acid in the cell or in the subject.

14. The method of any one of claims 4-6 and 10-13, wherein the expression level is based on mRNA level or protein level.

15. The method of any one of claims 4-6 and 10-14, wherein the expression level is increased by at least 10%, 15%, 20%, 25%, 30%, 35%, 40%, 45%, 50%, 60%, 70%, 80%, 90%, 100%, 120%, 150%, 180%, 200%, 220%, 250%, 280%, 300%, 400%, 500%, 600%, 700%, 800%, or 900%.

16. The method of any one of claims 1-6, and 10-15, wherein the expression level is determined within expression duration of the exogenous nucleic acid.

17. The method of any one of claims 7-9, wherein the expression duration is the period during which the exogenous nucleic acid is expressed at a detectable level or at a physiologically effective level.

18. The method of any one of claims 7-9, wherein the expression duration is prolonged by at least 1 day, 2 days, 3 days, 4 days, 5 days, 6 days, 7 days, 8 days, 9 days or 10 days.

19. The method of any one of claims 1-18, wherein the exogenous nucleic acid comprises or is contained within a viral vector, plasmid, or exosomes.

20. The method of claim 19, wherein the viral vector comprises an adeno-associated virus (AAV) vector.

21. The method of claim 20, wherein the AAV vector comprises an AAV virus particle.

22. The method of any one of claim 1-21 wherein the exogenous nucleic acid comprises an encoding sequence that encodes for a protein of interest or a portion thereof, or that encodes for a functional RNA or a portion thereof.

23. The method of claim 22, wherein the protein of interest comprises a therapeutic protein, an immunogenic protein, a reporter protein, a nuclease or a therapeutic target protein, and/or the functional RNA comprises an antisense oligonucleotide, ribozyme, RNAs that effect spliceosome-mediated/raw-splicing, interfering RNAs (RNAi), or other non-translated functional RNAs, such as guide RNAs and single guide RNAs.

24. The method of claim 23, wherein:
the therapeutic protein is selected from the group consisting of: SMN1, NAGLU, SGSH, IDS, FVIII, FIX, BTK, ABCD1, ACADVL, AR, HBB, SCN1A, CFTR, CSF2RA, IL2AG, PHA, STK11, PIGA, OTC, NAGS, DMPK, CNBP, ACADM, GNAS, FBN1, LIPA, SLC7A7, HADHA, GHR, IDV, ALPL, SLC25A15, HTT, HCS, NOTCH3, ALDOB, ATP7B, GAA, GCDH, SLC12A3, GBA, MEFV, GLA, CLCN1 NR0B1, ASS1, SLC25A13, SLC22A5, SCN5A, BTD, ACAT1, ARG1, CYP21A2, a chimeric antigen receptor (CAR), an antibody (e.g. monoclonal or bispecific or multi-specific), insulin, glucagon-like peptide-1, peptide hormones, growth factors, erythropoietin (EPO), cytokines, coagulation factors, antihemophilic factors, interferons, Fc-fusion proteins (such as CTLA-4 Fc-fusion, VEGFR Fc-fusion), and therapeutic enzymes (e.g. lysosomal hydrolase, and sulfatases);
the immunogenic protein is selected from the group consisting of an immunogenic protein from orthomyxovirus (e.g. influenza virus), lentivirus (e.g. HIV, SIV), arenavirus (Lassa fever virus), poxvirus (e.g. vaccinia), flavivirus (e.g. yellow fever virus), filovirus (Ebola virus), bunyavirus (RVFV, CCHF, or SFS viruses), coronavirus (e.g. SARS, MERS, or COVID-19), poliovirus, herpes virus (CMV, EBV, HSV), mumps virus, measles virus, rubella virus, diphtheria toxin, pertussis, and hepatitis virus (e.g. HAV, HBV, or HCV);
the nuclease comprises a Zinc-finger nucleases (ZFN), a transcription activator-like effector nucleases (TALEN), or a Cas family protein (such as Cas1, Cas1B, Cas2, Cas3, Cas4, Cas5, Cas6, Cas7, Cas8, Cas9 (also known as Csn1 and Csx12), Cas 10, Cas 11, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologues thereof, or modified versions thereof,
the reporter proteins is selected from the group consisting of: a fluorescent protein (e.g., EGFP, GFP, RFP, BFP, YFP, or dsRED2), an enzyme that produces a detectable product, such as luciferase (e.g., from Gaussia, Renilla, or Pho firms), b-galactosidase, b-glucuronidase, alkaline phosphatase, chloramphenicol acetyltransferase gene, and proteins that can be directly detected;
the therapeutic target protein (e.g. CTLA-4, HER2, Nectin-4, Sclerostin, P-Selectin, VEGF, RSVF, VEGFR2, CD79, IL23p19, vWF, IFN-γ, C5, PD-1, PD-L1, CGRP, CD3, CD11a, CD20, CD22, CD30, CD33, CD38, CD40, CD52, IgE, KLK, CCR4, FGF-23, IL-6R, IL-5, IL-23p19, IL-2R, IL-17R, IL-17, CD4, FIX/FX, IL-12, IL-23, IL-1β, IL-5R, IL-6R, IL-4/IL-13, PDGF-α, Dabigatran, SLAMF7, EGFR, PCSK9, GD2, CD3, CD19, α4β7 integrin, α4β1 integrin, PA, BLyS, RANK, TNF-α, EpCAM, GGTA1, Endostatin, Angiostatin); or
the functional RNA modulates a biological target selected from the group consisting of: multiple drug resistance (MDR) protein target, a tumor target (e.g. VEGF, Her2, EGFR, PD-L1 and so on), a pathogen target such as a viral surface antigen (e.g. hepatitis B surface antigen gene), a defective gene product (mutated dystrophins), or a therapeutic target (e.g. myostatin).

25. The method of any one of claims 22-24, wherein the encoding sequence is operably linked to one or more regulatory sequences.

26. A method of priming a subject having a condition treatable by an exogenous nucleic acid or the expression product thereof, the method comprising:
administering an effective amount of an aspirin compound to the subject prior to or concurrently with the delivery of the exogenous nucleic acid to the cell,
wherein the exogenous nucleic acid comprises double-stranded DNA, or can be converted to double-stranded DNA in the cell after delivery.

27. A method of treating or preventing a condition treatable or preventable by an exogenous nucleic acid or the expression product thereof, the method comprising:
delivering to the subject a therapeutically effective amount of the exogenous nucleic acid,
wherein the exogenous nucleic acid comprises double-stranded DNA, or can be converted to double-stranded DNA in the subject after delivery, and
wherein the subject has been or is concurrently being administered with an aspirin compound.

28. A method of treating or preventing a condition treatable or preventable by an exogenous nucleic acid or the expression product thereof, the method comprising:
a) administering an effective amount of an aspirin compound to the subject; and
b) delivering to the subject a therapeutically effective amount of the exogenous nucleic acid,
wherein the step a) is prior to or concurrently with the step b),
wherein the exogenous nucleic acid comprises double-stranded DNA, or can be converted to double-stranded DNA in the subject after delivery.

29. A method of reducing adverse effects or improving tolerance to an exogenous nucleic acid in a subject, the method comprising:
delivering to the subject a sub-therapeutic amount of the exogenous nucleic acid for treating or preventing a condition,
wherein the exogenous nucleic acid comprises double-stranded DNA, or can be converted to double-stranded DNA in the subject after delivery, and
wherein the subject has been or is concurrently being administered with an effective amount of an aspirin compound.

30. A method of reducing adverse effects or improving tolerance to an exogenous nucleic acid in a subject, the method comprising:
a) administering an effective amount of an aspirin compound to the subject; and
b) delivering to the subject a sub-therapeutic amount of the exogenous nucleic acid for treating or preventing a condition,
wherein the step a) is prior to or concurrently with the step b),
wherein the exogenous nucleic acid comprises double-stranded DNA, or can be converted to double-stranded DNA in the subject after delivery.

31. The method of any one of claims 26-30, wherein the exogenous nucleic acid comprises or is contained within a viral vector, plasmid, or exosomes.

32. The method of claim 31, wherein the viral vector comprises an adeno-associated virus (AAV) vector.

33. The method of claim 32, wherein the AAV vector comprising an AAV virus particle.

34. The method of any one of claim 26-33 wherein the exogenous nucleic acid comprises an encoding sequence that encodes for a protein of interest or a portion thereof, or that encodes for a functional RNA or a portion thereof.

35. The method of claim 34, wherein the protein of interest comprises a therapeutic protein, an immunogenic protein, a reporter protein, a nuclease or a therapeutic target protein, and/or the functional RNA comprises an antisense oligonucleotide, ribozyme, RNAs that effect spliceosome-mediated/raw-splicing, interfering RNAs (RNAi), or other non-translated functional RNAs, such as guide RNAs and single guide RNAs.

36. The method of claim 34, wherein:
the therapeutic protein is selected from the group consisting of: SMN1, NAGLU, SGSH, IDS, FVIII, FIX, BTK, ABCD1, ACADVL, AR, HBB, SCN1A, CFTR, CSF2RA, IL2AG, PHA, STK11, PIGA, OTC, NAGS, DMPK, CNBP, ACADM, GNAS, FBN1, LIPA, SLC7A7, HADHA, GHR, IDV, ALPL, SLC25A15, HTT, HCS, NOTCH3, ALDOB, ATP7B, GAA, GCDH, SLC12A3, GBA, MEFV, GLA, CLCN1 NR0B1, ASS1, SLC25A13, SLC22A5, SCN5A, BTD, ACAT1, ARG1, CYP21A2, a chimeric antigen receptor (CAR), an antibody (e.g. monoclonal or bispecific or multi-specific), insulin, glucagon-like peptide-1, peptide hormones, growth factors, erythropoietin (EPO), cytokines, coagulation factors, antihemophilic factors, interferons, Fc-fusion proteins (such as CTLA-4 Fc-fusion, VEGFR Fc-fusion), and therapeutic enzymes (e.g. lysosomal hydrolase, and sulfatases);
the immunogenic protein is selected from the group consisting of an immunogenic protein from orthomyxovirus (e.g. influenza virus), lentivirus (e.g. HIV, SIV), arenavirus (Lassa fever virus), poxvirus (e.g. vaccinia), flavivirus (e.g. yellow fever virus), filovirus (Ebola virus), bunyavirus (RVFV, CCHF, or SFS viruses), coronavirus (e.g. SARS, MERS, or COVID-19), poliovirus, herpes virus (CMV, EBV, HSV), mumps virus, measles virus, rubella virus, diphtheria toxin, pertussis, and hepatitis virus (e.g. HAV, HBV, or HCV);
the nuclease comprises a Zinc-finger nucleases (ZFN), a transcription activator-like effector nucleases (TALEN), or a Cas family protein (such as Cas1, Cas1B, Cas2, Cas3, Cas4, Cas5, Cas6, Cas7, Cas8, Cas9 (also known as Csn1 and Csx12), Cas 10, Cas 11, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologues thereof, or modified versions thereof,
the reporter proteins is selected from the group consisting of: a fluorescent protein (e.g., EGFP, GFP, RFP, BFP, YFP, or dsRED2), an enzyme that produces a detectable product, such as luciferase (e.g., from Gaussia, Renilla, or Pho firms), b-galactosidase, b-glucuronidase, alkaline phosphatase, chloramphenicol acetyltransferase gene, and proteins that can be directly detected;
the therapeutic target protein (e.g. CTLA-4, HER2, Nectin-4, Sclerostin, P-Selectin, VEGF, RSVF, VEGFR2, CD79, IL23p19, vWF, IFN-γ, C5, PD-1, PD-L1, CGRP, CD3, CD11a, CD20, CD22, CD30, CD33, CD38, CD40, CD52, IgE, KLK, CCR4, FGF-23, IL-6R, IL-5, IL-23p19, IL-2R, IL-17R, IL-17, CD4, FIX/FX, IL-12, IL-23, IL-1β, IL-5R, IL-6R, IL-4/IL-13, PDGF-α, Dabigatran, SLAMF7, EGFR, PCSK9, GD2, CD3, CD19, α4β7 integrin, α4β1 integrin, PA, BLyS, RANK, TNF-α, EpCAM, GGTA1, Endostatin, Angiostatin); or
the functional RNA modulates a biological target selected from the group consisting of: multiple drug resistance (MDR) protein target, a tumor target (e.g. VEGF, Her2, EGFR, PD-L1 and so on), a pathogen target such as a viral surface antigen (e.g. hepatitis B surface antigen gene), a defective gene product (mutated dystrophins), or a therapeutic target (e.g. myostatin).

37. The method of any one of claims 35-36, wherein the sequence encoding the protein of interest is operably linked to one or more regulatory sequences.

38. The method of any one of claims 26-37, wherein the condition is characterized by deficiency of one or more functional genes or functional protein(s).

39. The method of claim 38, wherein the condition is a single gene disorder.

40. The method of claim 39, wherein the single gene disorder is a autosomal dominant, autosomal recessive, X-linked, Y-linked or mitochondrial.

41. The method of any one of claims 26-40, wherein the condition is selected from the group consisting of: Brugada Syndrome, Myotonic Dystrophy 1, Myotonic Dystrophy 2, Hereditary Multy-infarct Dementia, Huntington's disease, neurofibromatosis type 1, neurofibromatosis type 2, Marfan syndrome, Familial hypercholesterolemia (FH), Polycystic kidney disease, Hereditary spherocytosis, hereditary nonpolyposis colorectal cancer, hereditary multiple exostoses, Tuberous sclerosis, Von Willebrand disease, and acute intermittent porphyria), Dravet Syndrome, Peutz-Jeghers Syndrome, achondroplasia, Primary combined immune deficiency, Familial Adenomatous Polyposis (FAP), Spinocerebellar Ataxia, Multiple Endocrine Neoplasia, Beta-Ketothiolase deficiency, Biotinidase Deficiency, Hepatolenticular Degeneration, Spinal Muscular Atrophy, N-acetylglutamate synthase deficiency, Lysosomal Acid Lipase Deficiency, Lysinuric Protein Intolerance, Long Chain 3-hydroxyacyl-CoA Dehydrogenase Deficiency, Laron syndrome, Isovaleric Acidemia, Hyperornithinemia-hyperammonemia-homocitrullinuria syndrome, Holocarboxylase Synthetase Deficiency, Hereditary Fructose Intolerance, Glycogen storage disease type II, Glutaric Acidemia Type I, Gitelman Syndrome, Gaucher Disease, Familial Mediterranean Fever, Myotonia Congenita, Citrullinemia I, Citrullinemia II, Primary Carnitine Deficiency, Arginase Deficiency, Medium-chain acyl-CoA dehydrogenase deficiency, Sickle cell anaemia, cystic fibrosis, Tay-Sachs disease, Phenylketonuria, Lysosomal acid lipase deficiency, Glycogen storage diseases, Galactosemia, Niemann-Pick disease, spinal muscular atrophy (SMA), Roberts syndrome, Very long-chain acyl-CoA dehydrogenase deficiency, Pulmonary Cystic Fibrosis, Gaucher Disease, Werner Syndrome, Fanconi Anemia, Mucopolysaccharidosis (I, IIIA, IIIB, IVA, IVB, VI, VII, IX), Fragile X syndrome, Congenital Adrenal Hypoplasia, Duchenne muscular dystrophy, and Hemophilia A, Hemophilia B, Fabry Disease, X-linked agammaglobulinemia, X-linked Adrenoleukodystrophy, Spinal and bulbar muscular atrophy, Ornithine Transcarbamylase Deficiency, and Mucopolysaccharidosis II, Adrenoleukodystrophy (ALD), Chronic granulomatous disease, McCune-Albright syndrome, Paroxysmal Nocturnal Hemoglobinuria, ADA Imune deficiency, Amyotrophic lateral sclerosis (ALS), glucose-galactose, muscular dystrophy, Azoospermia, Ehlers-Danlos Syndrome, Retinitis Pigmentosa, Hemochromatosis, Melanoma, Retinoblastoma, Alzheimer Disease, Amyloidosis, Myotonic Dystrophy, giant axonal neuropathy, Alpha-1 antitrypsin, Parkinson's disease, Severe Combined Immunodeficiency (ADA-SCID/X-SCID), Heart disease, Cancer (e.g. leukemia, particularly, acute lymphoblastic leukemia), Diabetes, Schizophrenia and Alzheimer's disease.

42. The method of any one of claim 26-37, wherein the condition is Central Nervous System (CNS) disorder.

43. The method of claim 42, wherein the CNS disorder is selected from the group consisting of Parkinson's disease, Alzheimer's disease, Mucopolysaccharidosis type II, Mucopolysaccharidosis type IIA, Mucopolysaccharidosis type HIB, Huntington disease, amyotrophic lateral sclerosis, Epilepsy, Batten Disease, Spinocerebellar Ataxia, spinal muscular atrophy, Canavan disease, and Friedreich's ataxia.

44. The method of any one of claim 26-43, wherein the exogeneous nucleic acid is administered via a systemic route.

45. The method of any one of claim 42-44, wherein the exogeneous nucleic acid comprises sequence encoding for a protein of interest or a portion thereof, wherein the protein of interest is selected from the group consisting of Tau, MeCP2, NGF, APOE, GDNF, SUMF, SGSH, AADC, CD, p53, ARSA arylsulfatase A, ABCD1, SMN1, NAGLU, SOD1, C9ORF72, TARDBP, FUS, HTT, LRRK2, PARIS, PARKIN, GAD, and α-synuclein.

46. The method of any one of claim 26-45, wherein the exogenous nucleic acid comprises an AAV vector of AAV9 serotype (e.g. an AAV virus particle of AAV9 serotype).

47. The method of any one of claims 26-28 and 31-46, wherein the therapeutically effective amount ranges from 10⁶ to 10¹⁴ vg/kg (vector genomes/kg), or is no more than 10¹⁴ vg/kg (e.g. no more than 10¹³ vg/kg, 10^{12.5} vg/kg, 10¹² vg/kg, 10¹¹ vg/kg or even lower).

48. The method of any one of claims 26-28 and 31-47, wherein the therapeutically effective amount is a sub-therapeutic amount.

49. The method of any of claims 29-30 and 48, wherein the sub-therapeutic amount is no more than 90%, no more than 80%, no more than 70%, no more than 60%, no more than 50%, no more than 40%, no more than 30%, no more than 20%, no more than 10%, no more than 5%, no more than 2%, or no more than 1% of the conventional amount of the same exogenous nucleic acid that would otherwise be required without the administration of the aspirin compound.

50. The method of claim 49, wherein the sub-therapeutic amount of the AAV vector or AAV virus particle is no more than 10⁷ vg/kg (vector genomes/kg), no more than 10⁸ vg/kg, no more than 10⁹ vg/kg, no more than 10¹⁰ vg/kg, no more than 10¹¹ vg/kg, no more than 10¹² vg/kg, no more than 10¹³ vg/kg, or no more than 10¹⁴ vg/kg.

51. The method of any one of claims 26-50, wherein the aspirin compound is administered to the subject at least 1 day, 2 days, 3 days, 4 days, 5 days, 6 days or 7 days prior to the delivery of the exogenous nucleic acid to the subject, and/or is administered for once or repetitively (e.g. twice, three times, four times and so on) prior to the delivery of the nucleic acid.

52. The method of any one of claims 26-51, wherein the aspirin compound and/or the exogenous nucleic acid is administered via parenteral, oral, enteral, buccal, nasal, topical, rectal, vaginal, transmucosal, epidermal, transdermal, dermal, ophthalmic, pulmonary, cardiac, subcutaneous, intraparenchymal, intracerebroventricular, intrathecal administration routes to the subject.

53. The method of any one of claims 26-52, wherein the aspirin compound is administered to the subject at an amount of no more than 30 mg/kg, no more than 50 mg/kg, no more than 100 mg/kg, no more than 110 mg/kg, no more than 120 mg/kg, no more than 120 mg/kg, no more than 130 mg/kg, no more than 140 mg/kg, no more than 150 mg/kg, no more than 160 mg/kg, no more than 170 mg/kg, no more than 180 mg/kg, no more than 190 mg/kg, or no more than 200 mg/kg.

54. A pharmaceutical composition comprising a sub-therapeutic amount of an exogenous nucleic acid and a pharmaceutically acceptable carrier, optionally further comprising an aspirin compound, wherein the exogenous nucleic acid comprises double-stranded DNA, or can be converted to double-stranded DNA in a cell after delivery of the exogenous nucleic acid to the cell.

55. A pharmaceutical composition comprising an exogenous nucleic acid, an aspirin compound, and a pharmaceutically acceptable carrier, wherein the exogenous nucleic acid comprises double-stranded DNA, or can be converted to double-stranded DNA in a cell after delivery of the exogenous nucleic acid to the cell.

56. The pharmaceutical composition of claim 54 or 55, further comprising an instruction for use that indicates the aspirin compound is to be administered prior to or concurrently with administration of the pharmaceutical composition.

57. The pharmaceutical composition of any one of claims 55-56, wherein the exogenous nucleic acid comprises or is contained within a viral vector, plasmid, or exosomes.

58. The pharmaceutical composition of claim 57, wherein the viral vector comprises an adeno-associated virus (AAV) vector.

59. The pharmaceutical composition of claim 58, wherein the AAV vector comprising an AAV virus particle.

60. The pharmaceutical composition of any one of claim 54-59, wherein the exogenous nucleic acid comprises a sequence encoding for a protein of interest, or a portion thereof.

61. The pharmaceutical composition of claim 60, wherein the sequence encoding the protein of interest is operably linked to one or more regulatory sequences.

62. The pharmaceutical composition of any one of claims 59-61, wherein the pharmaceutical composition is in a unit dose, and contains no more than 10¹⁰ vg, 10^{10.5} vg, 10¹¹ vg, 10^{11.5} vg, 10¹² vg, 10^{12.5} vg, 10¹³ vg, 10^{13.5} vg, 10¹⁴ vg, 10^{14.5} vg, 10¹⁵ vg, 10^{15.5} vg, or 10¹⁶ vg of AAV virus particle.

63. A kit comprising:
a) a first composition comprising an aspirin compound; and
b) a second composition comprising an exogenous nucleic acid,
wherein the exogenous nucleic acid comprises double-stranded DNA, or can be converted to double-stranded DNA in a cell after delivery of the exogenous nucleic acid to the cell.

64. The kit of claim 63, further comprising an instruction for use that indicates that the first composition is to be administered prior to or concurrently with the second composition.

65. The kit of claim 63, wherein the first composition and the second composition can be readily mixed to provide a combined composition before use.

66. The kit of claim 63, wherein the second composition comprises the exogenous nucleic acid at a sub-therapeutic amount.

67. A kit comprising a composition comprising an aspirin compound and an exogenous nucleic acid, wherein the exogenous nucleic acid comprises double-stranded DNA, or can be converted to double-stranded DNA in a cell after delivery of the exogenous nucleic acid to the cell.

68. A composition comprising:
an aspirin compound and an exogenous nucleic acid in combination, wherein the exogenous nucleic acid comprises double-stranded DNA, or can be converted to double-stranded DNA in a cell after delivery of the exogenous nucleic acid to the cell.


SEQUENCE LISTING (11 sequences)

SEQ ID NO: 1 - DNA, 287 bp, Artificial Sequence
  Synthetic
ctgtttactctggttaatttttaaaggagggtaaacagtgcctgaaagctgacctttgcccacattcctccggtagacattaacttattaaattgattctgattacaaatctgacctttgcccccatctcacccagtaacaatgcaagagttgatgtcagtctataaaaagcgaagcgcgcggtgggcggggttcgctgcctgcaggtgagtatctcagggatccagacatggggatatgggaggtgcctctgatcccagggctcactgtgggtctctctgttcaca

SEQ ID NO: 2 - DNA, 558 bp, Artificial Sequence
  Synthetic
atgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 3 - DNA, 718 bp, Artificial Sequence
  Synthetic
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcgggatcagccaccgcggtggcggcctagagtcgacgaggaactgaaaaaccagaaagttaactggtaagtttagtctttttgtcttttatttcaggtcccggatccggtggtggtgcaaatcaaagaactgctcctcagtggatgttgcctttacttctag

SEQ ID NO: 4 - DNA, 2217 bp, Artificial Sequence
  Synthetic
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 5 - DNA, 2211 bp, Artificial Sequence
  Synthetic
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 6 - DNA, 2214 bp, Artificial Sequence
  Synthetic
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctggagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccagtacctgtattacctgaacagaactcagaatcagtccggaagtgcccaaaacaaggacttgctgtttagccgtgggtctccagctggcatgtctgttcagcccaaaaactggctacctggaccctgttaccggcagcagcgcgtttctaaaacaaaaacagacaacaacaacagcaactttacctggactggtgcttcaaaatataacctcaatgggcgtgaatccatcatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgattttactgtggacaacaatggactttatactgagcctcgccccattggcacccgttacctcacccgtcccctgtaa

SEQ ID NO: 7 - DNA, 1653 bp, Homo sapiens
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 8 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 737 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 11 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP